(19) United States
(12) Patent Application Publication
(10) Pub. No.: US 2023/0165934 A1
(43) Pub. Date: Jun. 1, 2023

(54) MEANS AND METHODS FOR TREATING COPPER-RELATED DISEASES

(71) Applicants: HELMHOLTZ ZENTRUM MÜNCHEN - DEUTSCHES FORSCHUNGSZENTRUM FÜR GESUNDHET UND UMWELT (GMBH), NEUHERBERG (DE); THE REGENTS OF THE UNIVERITY OF MICHIGAN, ANN ARBOR, MI (US); IOWA STATE UNIVERSITY RESEARCH FOUNDATION, INC., AMES, IA (US)

(72) Inventors: Hans ZISCHKA, Munich (DE); Josef LICHTMANNEGGER, Neuching (DE); Alan Angelo DISPIRITO, Ames, IA (US); Jeremy David SEMRAU, Ann Arbor, MI (US)

(21) Appl. No.: 18/101,233

(22) Filed: Jan. 25, 2023

Related U.S. Application Data
(63) Continuation-in-part of application No. 17/221,675, filed on Apr. 2, 2021.
Application No. 17/221,675 is a division of application No. 16/063,220, filed on Jun. 15, 2018, now Pat. No. 11,000,568.

(30) Foreign Application Priority Data
Dec. 18, 2015 (EP) 15201070.8
Feb. 19, 2016 (LU) 92979

(51) Int. Cl. A61K 38/16; A61P 1/16

(52) U.S. Cl. CPC A61K 38/164; A61P 1/16

(57) ABSTRACT

The present invention relates to the field of (bio-)medicine, and more particularly to the treatment of copper-related diseases. Novel means and methods for depleting (excess) copper from organs and/or the circulation are provided. Agents with a high copper binding affinity and stabilized forms thereof are provided, as well as a novel treatment regimen. The means and methods of the present invention are particularly useful for treatment of Wilson Disease, but also for treatment of other conditions.

### CROSS-REFERENCES TO RELATED APPLICATIONS

This application is a continuation-in-part of U.S. patent application Ser. No. 17/221, 675, filed on Apr. 2, 2021, which is a divisional application of U.S. patent application Ser. No. 16/063,220 filed on Dec. 16, 2016, which is U.S. national phase application of International PCT Patent Application No. PCT/EP2016/081407, which was filed on Dec. 16, 2016, which claims priority to Luxembourg Application No. 92979, filed Feb. 19, 2016, and European Application No. 15201070.8, filed Dec. 18, 2015. These applications are incorporated herein by reference in their entireties.

### GOVERNMENT STATEMENT OF INTEREST

This invention was made with government support under grant DE-SC0006630 awarded by the U.S. Department of Energy. The government has certain rights in the invention.

### BACKGROUND

Copper is an essential trace element for eukaryotes and most prokaryotes that plays an important role in critical biological functions such as enzyme activity, oxygen transport and cell signaling. However, due to its high redox activity and its ability to catalyze the production of free radicals, copper can have detrimental effects on lipids, proteins, DNA and other biomolecules. Particularly, mitochondria are thought to be the major targets for oxidative damage resulting from copper toxicity. Moreover, copper can interfere with proteins and can displace other metals such as zinc from metalloproteins, thereby inhibiting their activity. In order to prevent copper from exerting its potentially toxic effects, it usually does not exist in free form, but only as a complex. In the human body, approximately 95% of the copper in plasma is bound to proteins such as ceruloplasmin, a multicopper ferroxidase that is synthesized and secreted by hepatocytes. It is estimated that less than 1 atom of free copper is present per cell.

Due to its ambivalent role in metabolism, any imbalance in copper bioavailability inevitably leads to deficiency or toxicity, and all organisms have evolved mechanisms that regulate its absorption, excretion and bioavailability. In mammals, copper absorption occurs in the small intestine via enterocyte uptake, followed by its transfer into the blood by the copper transporter ATP7A. The liver plays a critical role in copper metabolism, serving both as the site of copper storage and regulating its distribution to serum and tissues and excretion of excess copper into the bile. Particularly, hepatocytes transport and regulate physiological copper via the specialized transporter ATP7B.

ATP7A and ATP7B are closely related in structure and function, with approximately 60% amino acid sequence identity. They undergo ATP-dependent cycles of phosphorylation and dephosphorylation to catalyze the translocation of copper across cellular membranes for the metallation of many essential cuproenzymes, as well as for the removal of excess cellular copper to prevent toxicity.

ATP7B mutations result in a major impairment in the ability of hepatocytes to maintain copper homeostasis at the cellular and systemic levels, resulting in impaired biliary copper excretion and persistent copper accumulation in the liver, a condition known as Wilson disease (WD). This can lead -most likely due to the spillover of liver copper (Bandmann et al., The Lancet. Neurology 14, 103-113 (2015))- to deleterious effects on the brain and in many cases to chronic liver disease but also to fulminant hepatic failure (Gitlin, Gastroenterology 125, 1868-1877 (2003).

Untreated Wilson Disease is universally fatal, with most patients dying from liver disease. In order to restore body copper homeostasis, the clinically used copper chelators D-penicillamine (D-PA) and trientine (TETA) or the candidate drug tetrathiomolybdate (TTM) are administered daily (Gitlin J D, Gastroenterology. 2003 December; 125(6):1868-77). This lifelong therapy is effective only if commenced before the onset of advanced hepatic or neurologic disease (Roberts et al., Am J Clin Nutr 88, 851S-854S (2008)). The same holds true for zinc salts, which are primarily used in mild cases of WD to decrease copper absorption via the gastrointestinal tract or as copper maintenance therapy in chelator treated WD patients (Gitlin J D, loc. cit.). However, in circumstances of acute liver failure—caused by either delayed diagnosis, treatment failure, or rapidly developing fulminant hepatitis—death is almost certain unless liver transplantation is performed (Gitlin J D, loc. cit.). All of the currently FDA/EMA-approved copper chelators have severe adverse effects, including bone marrow toxicity, nephrotoxicity, hepatotoxicity, anemia and triggering of autoimmune disease (Gitlin J D, loc. cit.). Due to the toxicity of D-PA, discontinuation of treatment is required in almost one third of WD patients (Weiss & Stremmel, Current gastroenterology reports 14, 1-7 (2012)).

Currently approved pharmacological treatments usually fail to restore copper homeostasis in acute WD, thus rendering liver transplantation the only viable treatment option. Given these issues, there is a clear unmet medical need for an alternative and innovative treatment of WD and other copper-related disease. The technical problem underlying the present application is to comply with the unmet medical need for an alternative and innovative treatment of copper-related disease, such as WD, particularly acute WD.

### SUMMARY

The present inventors, for the first time, suggest—based on the unexpected capability of methanobactin to massively deplete copper to bring liver copper very close to normal physiological levels from hepatocytes and hepatocyte mitochondria—(1) a novel treatment regimen involving phases of copper depletion followed by phases of prolonged non-treatment, (2) a novel treatment of (previously difficult-to-treat or untreatable) acute phase Wilson Disease and (3) a stabilized form of methanobactin that retains the superior capabilities of unstabilized Methanobactin (and is thus suitable for use in accordance with the treatment regimen and medical indication set out above) but offers the benefit of increased stability at body temperature.

Methanobactins are low molecular mass copper-binding molecules produced by many methanotrophic bacteria and have been demonstrated to mediate copper acquisition from the environment (Semrau et al., 2010. FEMS Microbiol. Rev 34:496-531). Group I MBs, characterized by the methanobactin from Methylosinus trichosporium OB3b (MB-OB3b), typically have two oxazolone groups each with an associated thioamide and an internal disulfide bridge and Group II MBs, characterized by the methanobactin from Methylocystis sp. SB2 (ARBM101; synonymously called mb-SB2) hereafter termed ARBM101) that contains one oxazolone group with an associated thioamide and one pyrazinedione or imidazolone group with an associated thioamide as well as a sulfate group. For the first time, the present inventors have demonstrated that methanobactins hold considerable potential for treatment of a variety of copper-related diseases and conditions, and, due to their excellent copper binding affinities (Choi et al., 2006. Biochemistry 45: 1442-1453) and tolerance in vivo, are promising new agents for a massive and fast depletion of excess copper levels in patients in need thereof. Due to their beneficial properties, methanobactins are considered to be particularly useful for acute de-coppering therapy in Wilson Disease patients.

Thus, in a first aspect, the present invention relates to a method of treating Wilson Disease in a subject, wherein the treatment comprises a treatment cycle of (a) a first phase of methanobactin administration followed by (b) a second phase of non-treatment, wherein the second phase exceeds the first phase. Said first phase may last for a period of 1, 2, 3, 4, 5, 6, 7, 8, 9, 10 or more consecutive days, and may involve administration of methanobactin in single doses once daily, twice daily, three times daily, four times daily, every other day or continuously. The second phase may last for about at least 1 week, 2 weeks, 3 weeks, 4 weeks, 5 weeks, 6 weeks, 7, weeks, 8 weeks, 9 weeks, 10 weeks or more. At least one further treatment cycle may follow on the second phase of non-treatment of the treatment cycle. Particularly, treatment according to the method of the invention may comprise continuous treatment cycles.

In a further aspect, the present invention also relates to copper-binding methanobactin for use in a method of treatment of acute phase Wilson Disease in a subject.

In any event, the methanobactin for the uses of the invention may comprise or consist of the following general formula (I):

R¹—(X)₂₋₅—R² (I)

wherein

R1 and R2 are each a 5-membered heterocycle comprising N and associated with an enethiolate;

and each X is independently selected from any amino acid.

It is further contemplated that the methanobactin may be derived from bacteria, including methanotroph and non-methanotroph bacteria, such as Methylocystis spec., Methylosinus spec., Methylomicrobium spec. and Methylococcus spec. For instance, the methanobactin may be selected from (a) a Methylosinus trichosporium OB3b methanobactin (mb-OB3b) (b) a Methylocystis sp. SB2 methanobactin (mb-SB2), (c) a Methylococcus capsulatus Bath methanobactin (mb-Bath) (d) a Methylomicrobium album BG8 methanobactin (mb-BG8), (e) a Methylocystis sp. M methanobactin, (f) a Methylocystis hirsuta CSC1 methanobactin and (g) a Methylocystis rosea methanobactin (mb-rosea), (h) a Methylosinus sp. LW3 methanobactin (mb-LW3), (i) a Methylosinus sp. LW4 methanobactin (mb-LW4), (j) a Methylocystis sp. LW5 (mb-LW5), (k) a Methylosinus sp. PW1 methanobactin (mb-PW1), (I) a Methylocystis parvus OBBP methanobactin (mb-OBBP), (m) a Cupriavidus basiliensis B-8 methanobactin (mb-B-8), (n) a Pseudomonas extremaustralis 14-3 methanobactin (mb-14-3), (o) a Azospirillum sp. B510 methanobactin (mb-B510), (p) a Tistrella mobilis KA081020-065 (mb-mobilis) methanobactin and (q) a Comamonas composti DSM 21721 methanobactin (mb-21721).

The methanobactin for the uses of the invention is envisaged to bind copper, in particular Cu(I), with a K_{d} of 10⁻¹⁵ or less.

Said methanobactin may be complexing Zn(I) and/or Zn(II).

In a further aspect, the present invention provides a pharmaceutical composition comprising a stabilized methanobactin. Said pharmaceutical composition may be stable at 37° C. for at least 20 hours or more. Stabilization may be achieved by a) providing the pharmaceutical composition at a pH of ≥9 and/or by providing the methanobactin in the form of a complex with Zn(I) and/or Zn(II). Said zinc:methanobactin complex may be prepared by contacting an amount of Zn(I) and/or Zn(II) and an amount of methanobactin in a ratio of 1:1 in aqueous solution. The pharmaceutical composition of the present invention is envisaged to be useful for treating a variety of diseases, including Wilson Disease, cancer, neurodegenerative diseases, diabetes, bacterial infections, inflammatory diseases, fibrosis, cirrhosis, familiar amyotrophic lateral sclerosis, lead and/or mercury poisoning.

### DESCRIPTION OF THE FIGURES

FIG. 1: Liver disease in the LPP rat mirrors acute liver failure in WD patients by a devastating mitochondrial copper overload.

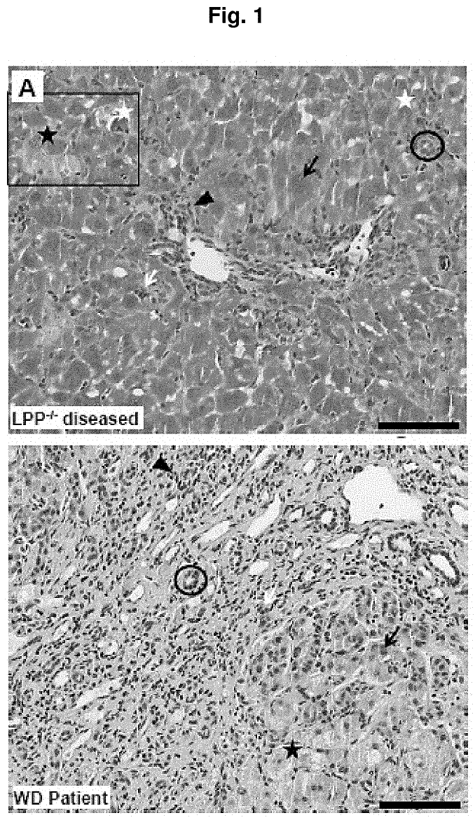

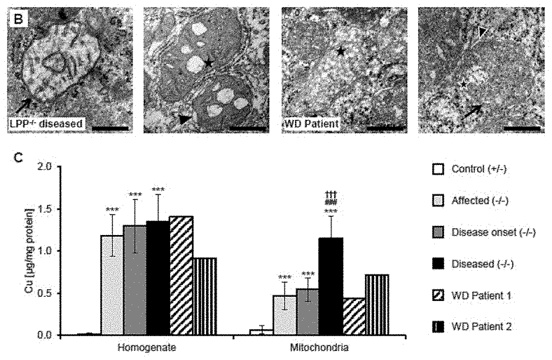

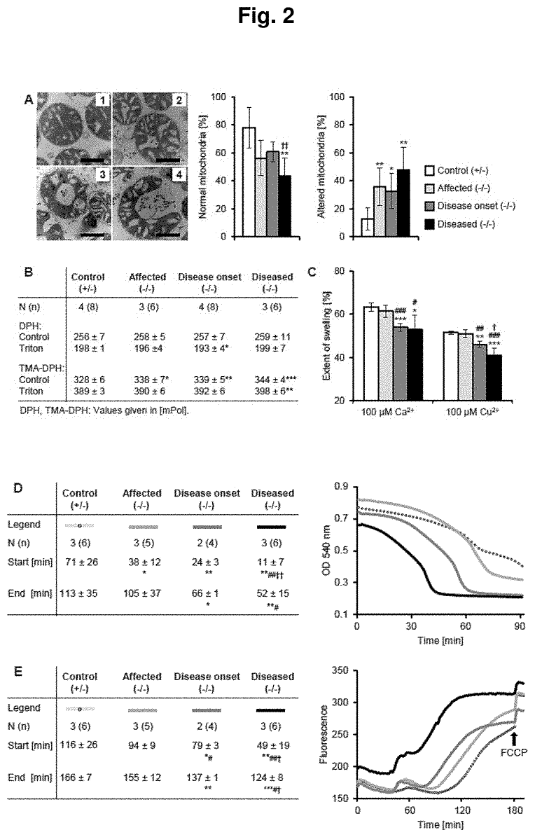

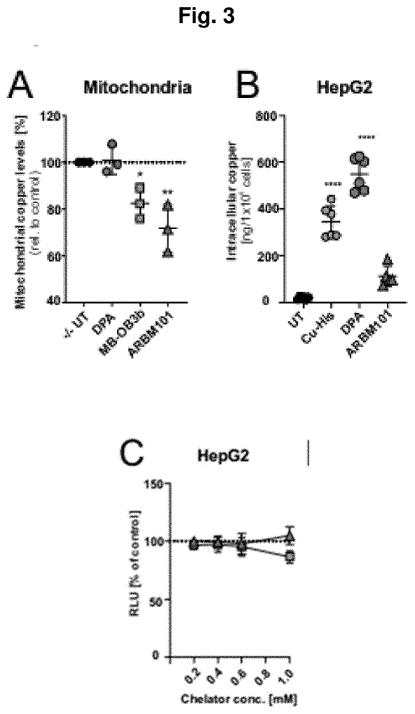

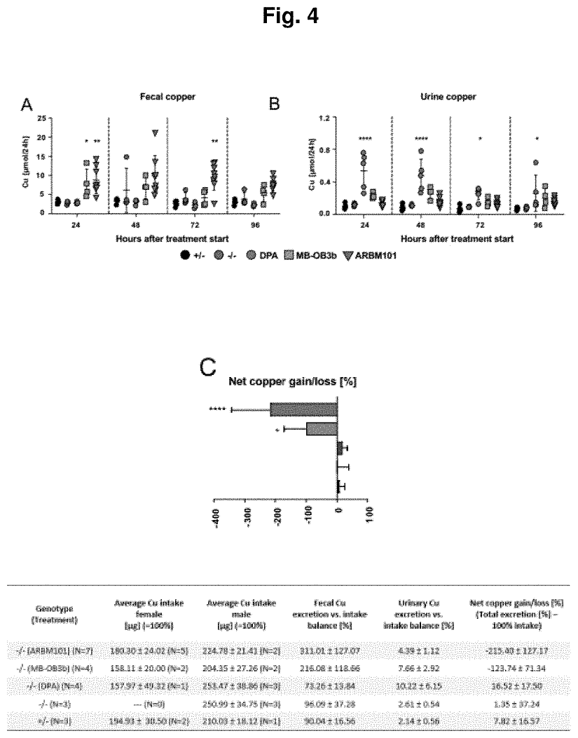

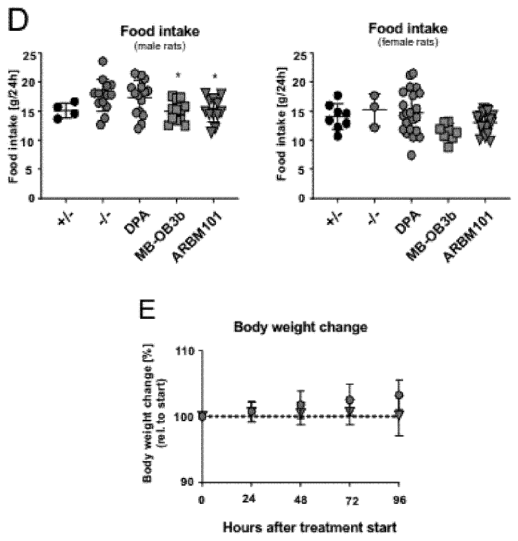

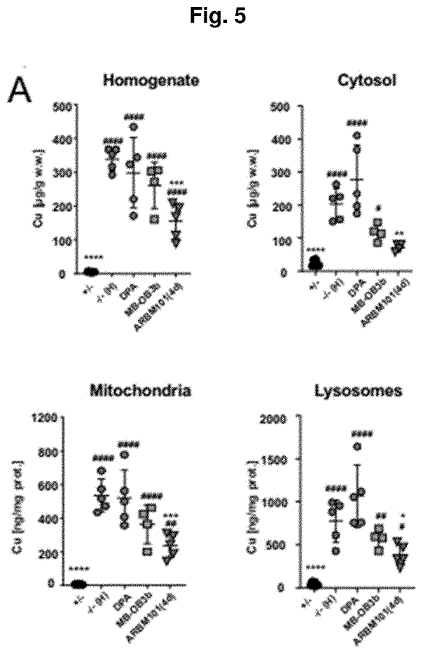

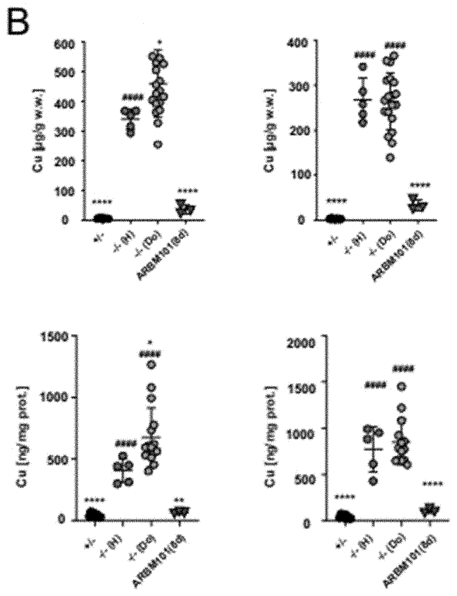

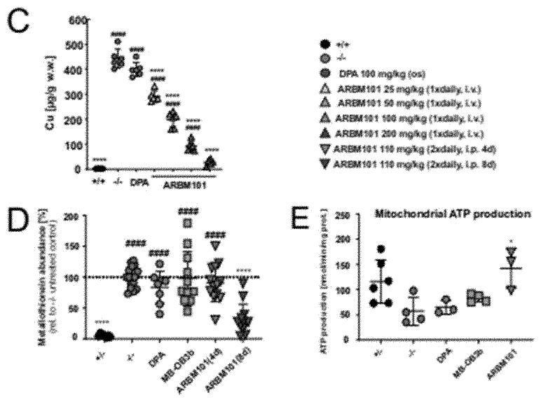

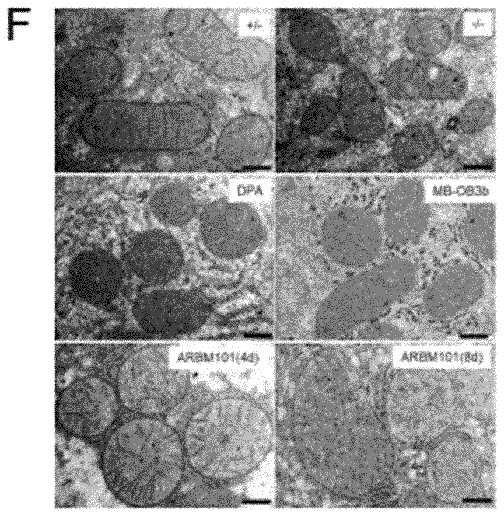

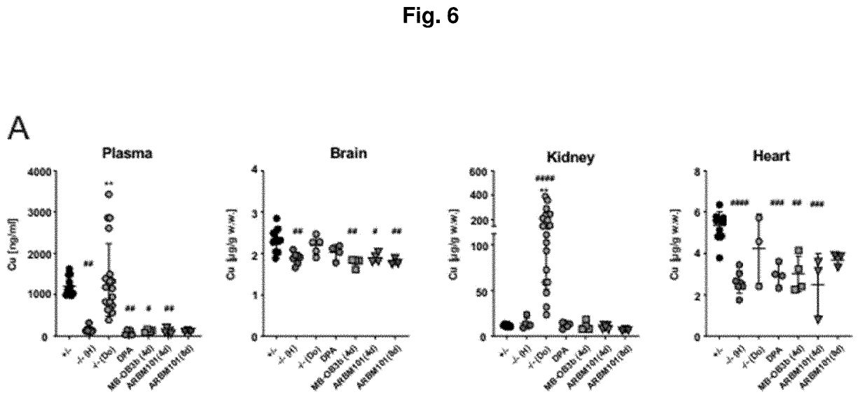

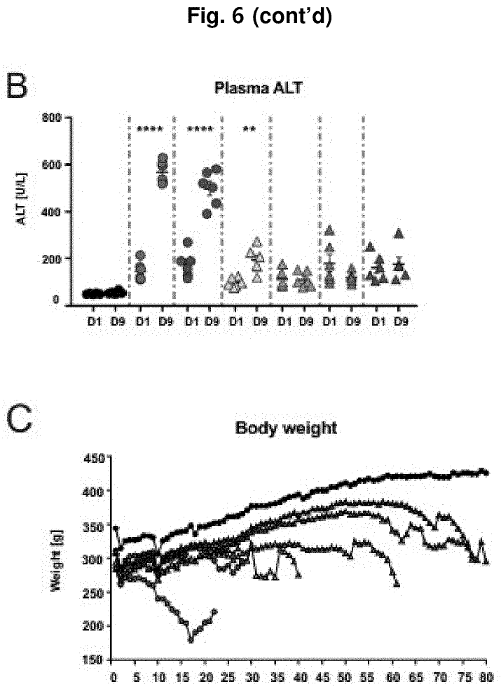

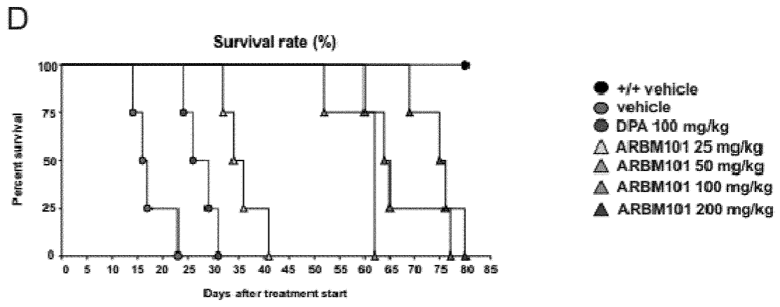

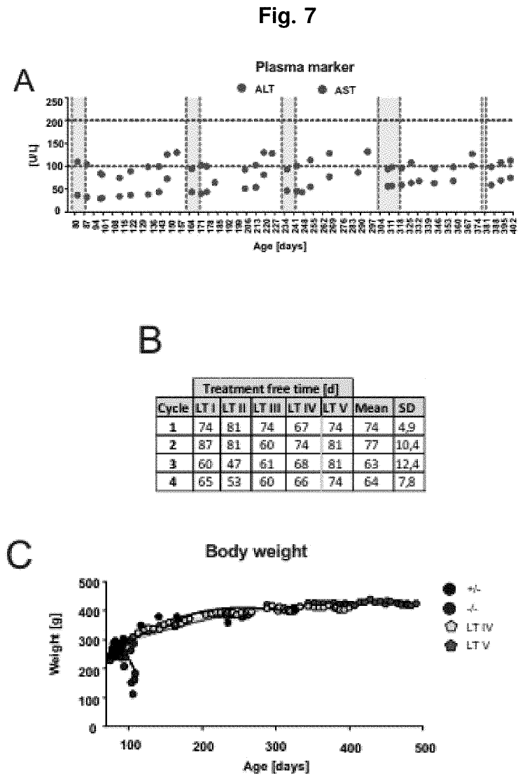

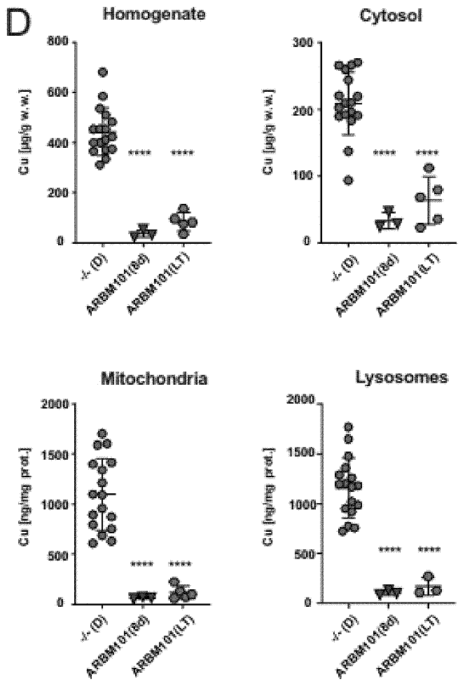

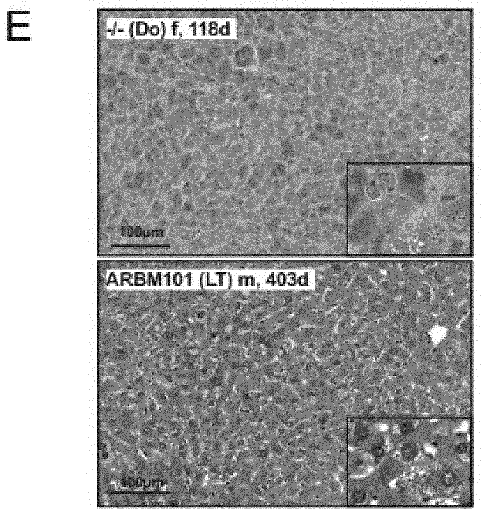

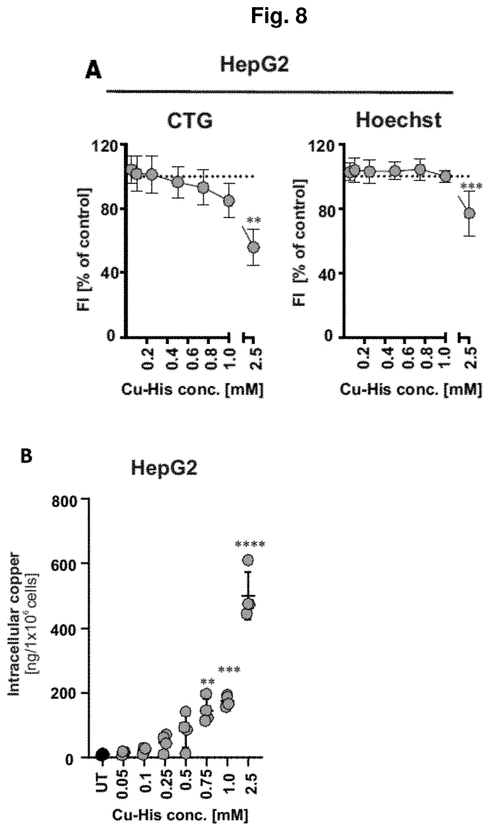

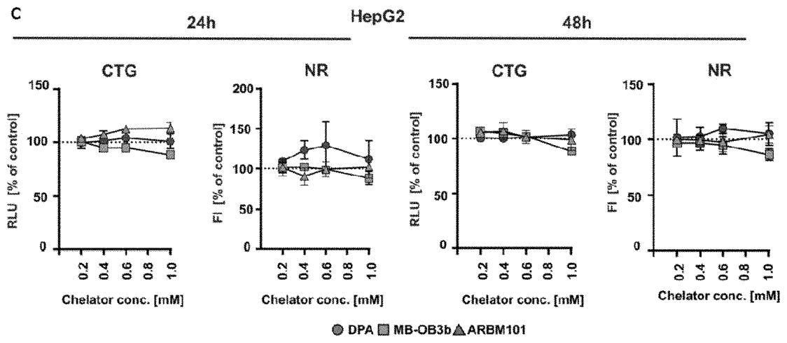

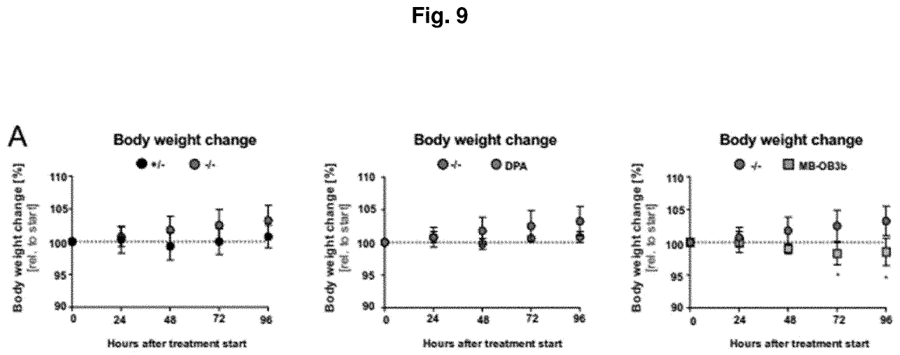

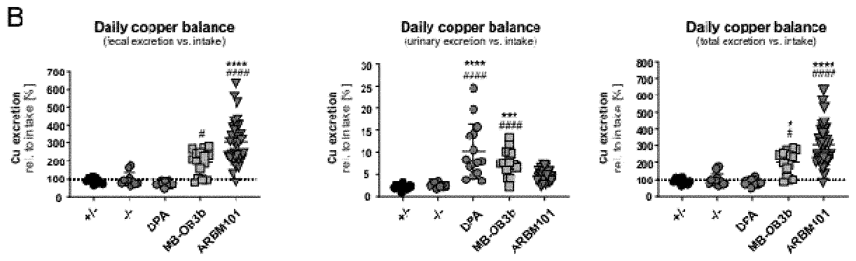

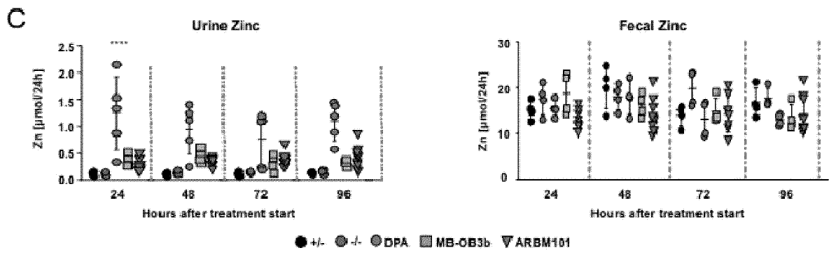

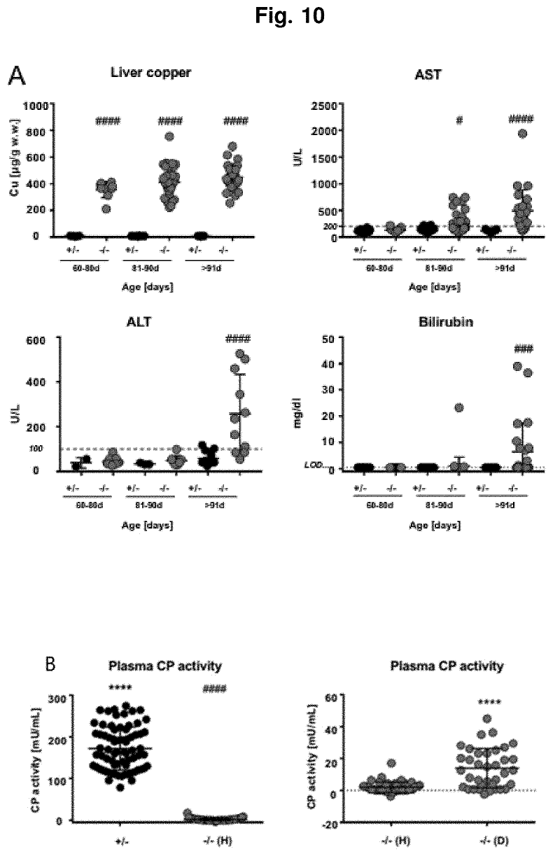

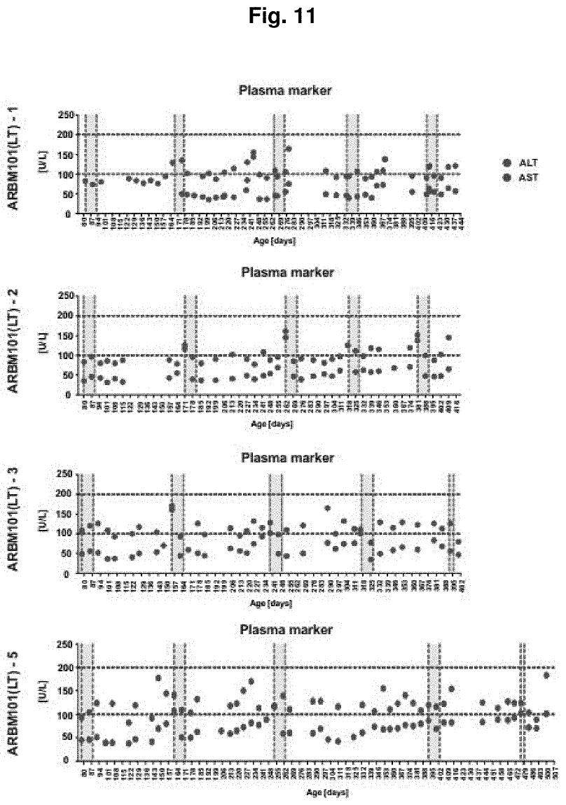

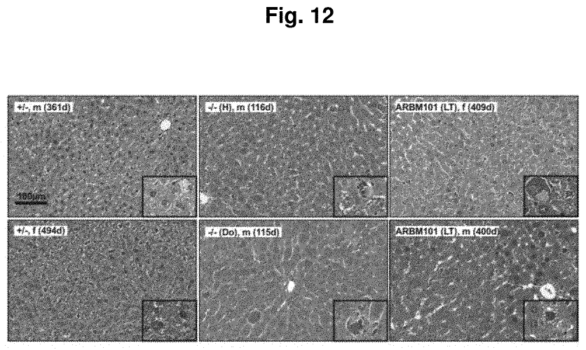

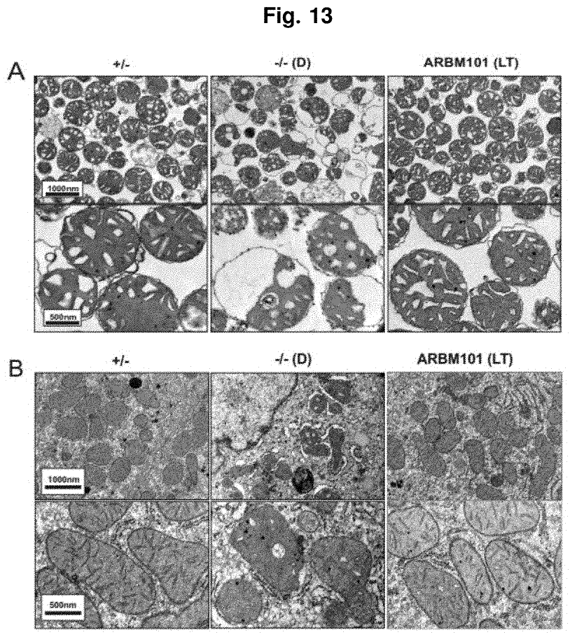

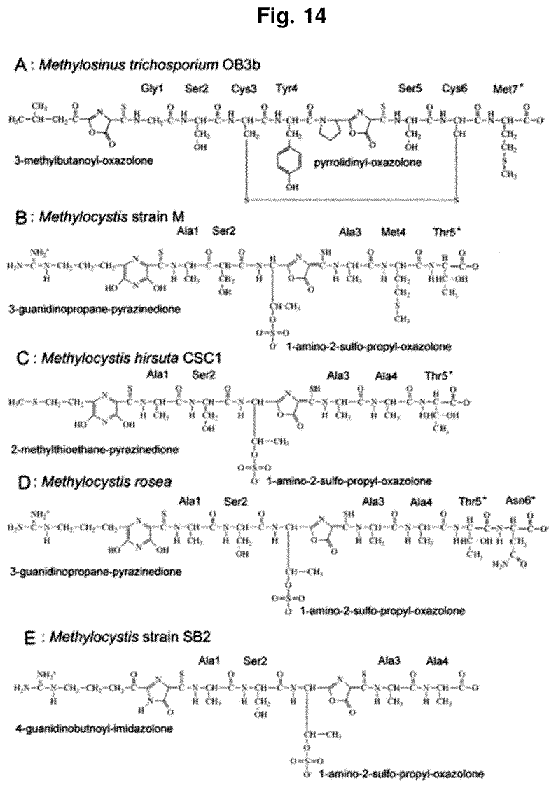

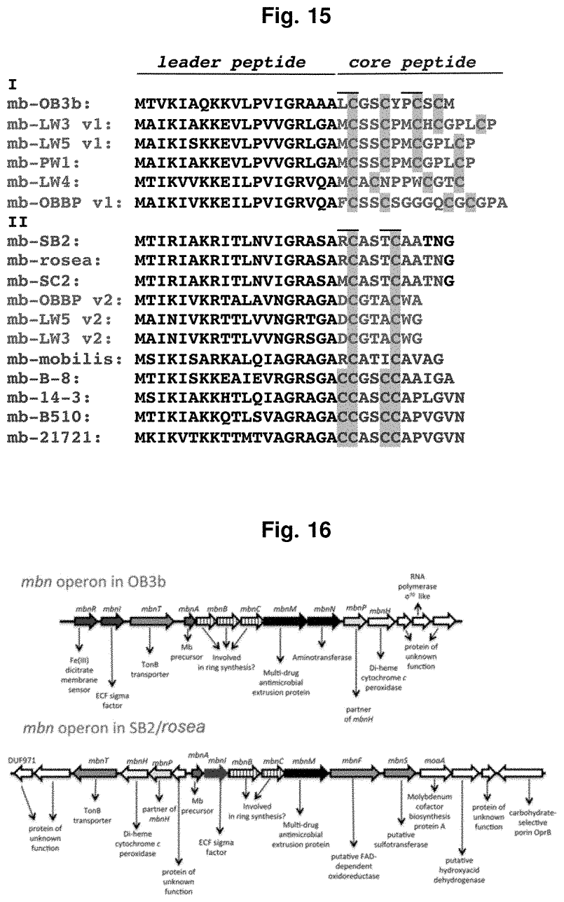

(A) Histopathological comparison (HE staining) of liver damage in diseased Atp7b^{-/-} LPP rats (hereafter LPP^{-/-} rats) (upper panel) and untreated WD patients with acute liver failure (lower panel). Tissue necrosis with resorptive inflammation as well as repair (fibrosis) is detectable (black arrowhead), proliferation of bile ducts (circle), anisokaryosis (black arrow), and several inflammatory infiltrations (white arrow) are marked. Insert shows apoptosis (white asterisk) and nodules with ballooned hepatocytes (black asterisk). Scale bar: 100 μm.

(B) Mitochondrial structure impairments in diseased LPP^{-/-} rats (both left panels) and untreated WD livers with acute liver failure (both right panels). Transparent vacuoles of varying sizes (asterisk), cristae dilations (arrow), marked differences in electron densities, and separated inner and outer membranes (arrowhead) can be identified. Scale bar: 500 nm.

(C) Comparable copper burden in whole liver homogenates and in purified liver mitochondria from LPN^{-/-} rats and untreated WD patients with acute liver failure. Control heterozygous LPP^{-/-} (N=17); affected LPP^{-/-} with strongly elevated copper but AST<200 U/L, bilirubin<0.5 mg/dl (N=13); disease onset LPP^{-/-} with AST<200 U/L, bilirubin<0.5 mg/dl (N=8); diseased LPP^{-/-} with AST>200 U/L, bilirubin>0.5 mg/dl (N=10). *Significant to control, ^{#}significant to affected, ^{\}significant to disease onset, *p<0.05, **p<0.01, ***p<0.001.

FIG. 2: Increasing copper load severely attacks the mitochondrial membrane integrity.

(A) The progressive disease states in LPP^{-/-} rats are paralleled by a decrease in normally structured mitochondria (type 1 and 2) and an increase in structurally altered mitochondria (type 3 and 4). Scale bar: 500 nm. Control LPP^{+/-} 82-89 d, N=4, n=766; affected LPP^{-/-} 82-93 d, N=6, n=886; disease onset LPP^{-/-} 81-93 d, N=4, n=784; diseased LPN^{-/-} 104-107 d, N=5, n=939. N=number of rats, n=number of analyzed mitochondria. *Significant to control, \significant to disease onset, *p<0.05, **p<0.01.

(B) Fluorescence polarization demonstration of physical alterations in mitochondrial membrane properties at the protein-lipid interface (TMA-DPH) but not at the membrane inner lipid phase (DPH) in LPN^{-/-} vs. control mitochondria. N=number of rats, n=number of measurements. *Significant to control, *p<0.05, **p<0.01, ***p<0.001.

(C) Upon calcium or copper induced MPT isolated LPP^{+/-} mitochondria undergo large amplitude swelling, which is significantly reduced in LPN^{-/-} mitochondria from diseased and disease onset rats. (N=2-3, n=4-6). *Significant to control, ^{#}significant to affected, ^{\}significant to disease onset, *p<0.05, **p<0.01, ***p<0.001.

(D) Calcium-induced (100 μM) MPT is induced significantly faster in mitochondria from diseased and disease onset LPP^{-/-} rats. Table (left) shows mean values and standard deviations whereas curves (right) depict one exemplary measurement. *Significant to control, ^{#}significant to affected, ^{\}significant to disease onset, *p<0.05, **p<0.01.

(E) LPP^{-/-} mitochondria lose their membrane potential at earlier time points compared to control mitochondria. Table (left) shows mean values and standard deviations whereas curves (right) depict one exemplary measurement. *Significant to control, ^{#}significant to affected, ^{\}significant to disease onset, *p<0.05, **p<0.01, **p<0.001.

FIG. 3: Methanobactins efficiently deplete excess copper both in vitro and in vivo.

(A) Isolated WD rat liver mitochondria were significantly copper depleted by MB-OB3b and ARBM101, but not by DPA (2 mM each). Copper levels are shown as % of buffer-treated WD rat mitochondria (N=3). Significance levels refer to buffer-treated WD rats *p≤0.05; **p≤0.005.

(B) Copper pre-loaded HepG2 cells (24 h, 0.5 mM CuHis) were copper depleted by ARBM101 in contrast to DPA (1 mM each, 24 h). Absolute copper values per 1×10⁶ cells are given (N=6). Significance refers to copper levels in untreated HepG2 cells (UT) **** p<0.0001 compared to UT.

(C) MB-OB3b (orange) and ARBM101 (green) did not present overt toxicity.

FIG. 4: Methanobactin-induced copper mobilization causes a negative daily copper balance.

Control and WD rats, the latter either untreated or i.p. DPA-, MB-OB3b- and ARBM101-treated, were housed in metabolic cages for four consecutive days (96 h). Significance levels refer to untreated control rats*p≤0.05; **p≤0.01; ****p≤0.0001.

(A) Methanobactins induced significant copper mobilisation into faeces (μmol/24 h), most sustainably for ARBM101 (N=4 and 7 for ARBM101).

(B) DPA routed minor copper amounts (μmol/24 h) into urine (N=4 and 7 for ARBM101).

(C) Methanobactins induced a significant net copper loss. The nutritive individual daily copper intake values were set to 100%. Individual daily copper fecal or urinary copper excretions were thereto given in percent with the net copper gain/loss calculated as total individual daily excretion minus 100%.

(D) Food consumption was followed in 24-hour periods separately for male and female rats (+/−N=3; −/−N=4; DPA N=4; MB-OB3b N=4; ARBM101 N=8).

(E) A 4-days ARBM101treatment (green) did not induce body weight loss (followed in 24-hour periods) versus untreated WD rats (blue) ((N=9).

FIG. 5: Methanobactin treatments deplete excessive liver copper.

WD rats were i.p. D-PA-, MB-OB3b- and ARBM101-treated. Significance refers to either +/− untreated control rats #p≤0.05; ##p≤0.005, ####p≤0.0001 or to −/− untreated WD rats*p≤0.05, **p≤0.005, ***p≤0.001, ****p≤0.0001.

(A) 4 days consecutive twice daily ARBM101 treatment significantly reduce whole-liver and liver subfractional cytosolic, mitochondrial and lysosomal copper levels and tendentious reductions were observed for MB-OB3b, but no changes for DPA. (+/−N=9;−/−H [still healthy] N=5; DPA N=5; MB-OB3b N=4; ARBM101 N=5).

(B) Whole liver and liver subfractional cytosolic, mitochondrial and lysosomal copper levels were depleted close to or down to control rat levels by a total of 8 days of twice daily ARBM101 treatment (+/−N=9; −/−H [still healthy] N=5; −/−Do [disease onset] N=14-19; ARBM101 (8d) N=3).

(C) ARBM101 dose-dependently reduced liver copper in WD rats (once daily i.v. treatments for 9 consecutive days, N=6 per group).

(D) 8 days of twice daily ARBM101 treatment significantly lowered hepatic metallothionein abundance as quantified by Immuno-blotting (+/−n=7;−/−H [still healthy] n=16; DPA n=8, MB-OB3b n=11; ARBM101 (4d) n=13; ARBM101 (8d) n=12).

(E) Mitochondrial ATP production capacity (in nmol/min/mg protein) was restored by a four-day twice daily ARBM101 treatment, tendentiously improved by MB-OB3b, but not by DPA (+/−N=6; −/−N=4; DPA N=3; MB-OB3b N=4; ARBM101 (4d) N=3).

(F) Electron micrographs of liver in-situ. Mitochondria from untreated WD rats demonstrated membrane detachments and dilated cristae (arrows), not restored by four days DPA treatment but by either four or eight days ARBM101 treatment (Scale bar: 500 nm).

FIG. 6: Methanobactin treatments do not deplete essential systemic copper but ensure prolonged animal survival with normal body weights.

(A) Copper levels in serum, brain, kidney and heart of untreated control, untreated still healthy (H) and untreated WD rats with beginning signs of hepatitis (Do), and treated WD rats. (Serum: +/−N=11; −/−(H) N=7; −/− (Do) N=5; DPA treated N=4; 4 d MB-OB3b N=4; 4 d ARBM101 N=4; 8 d ARBM101 N=3; Brain: +/−N=11; −/− (H) N=7; −/−(Do) N=5; DPA treated N=4; 4 d MB-OB3b N=4; 4 d ARBM101 N=4; 8 d ARBM101 N=3; Kidney: +/−N=9; −/−(H) N=5; −/−(Do) N=20; DPA treated N=5; 4 d MB-OB3b N=4; 4 d ARBM101 N=5; 8 d ARBM 101 N=3; Heart: +/−N=11; −/−(H) N=7; −/−(Do) N=3; DPA treated N=4; 4 d MB-OB3b: N=4; 4 d ARBM101 N=3; 8 d ARBM101 N=3).

(B) 9 consecutive days of medium and elevated dosed ARBM101 reduce signs of beginning hepatitis (serum ALT determined at treatment start and day 9 of treatment) in WD rats (N=6 per group).

(C) Body weight development in control and WD rats either vehicle-, DPA- or ARBM101-treated, the latter at increasing doses, for 9 consecutive days.

(D) Survival rates of WD rats treated for 9 days with either vehicle, 100 mg/kg DPA or increasing ARBM101doses, along with control animals (N=4).

FIG. 7: A new/novel treatment regimen in WD rats.

WD rats were repeatedly ARBM101 treated, typically twice daily for 8 days, followed by intermediate long treatment pauses.

(A) Exemplary Serum AST (green) and ALT (red) levels of one long-term ARBM101 treated WD rat. Treatment cycles are delimited by vertical dashed green lines. Thresholds indicative for disease onset are given as dashed horizontal lines (AST>200 U/L and ALT>100 U/L).

(B) Duration (days) of treatment pause for five ARBM101 long-term treated (LT) WD rats.

(C) Body weight curves of male control, untreated and two long-term ARBM101 treated WD rats.

(D) Whole liver and liver subfractional cytosolic, mitochondrial and lysosomal copper levels of 5 cycle long-term treated WD rats three weeks after the last treatment cycle (−/− diseased (D) N=17; 8 d ARBM101 N=3 (i.e. one cycle); long-term ARBM101 N=5 (3 for lysosomes)). Significance level ****p≤0.0001.

(E) Representative HE-stained formalin fixed paraffin embedded (FFPE) liver tissue sections from a WD rat at initial hepatitis stage and a long-term ARBM101 treated WD rat.

FIG. 8

(A) Cell viability of HepG2 cells were measured with the CellTiter-Glo® Luminescent Cell Viability Assay (CTG) and Hoechst assay 48 h after CuHis exposure. Untreated cells were used as control (100%). Mean±SD are given (N=6).

(B) Cu levels in HepG2 cells upon 48 h exposure to increasing copper (CuHis) concentrations. Mean±SD are given (N=4).

(C) ARBM101, MB-OB3b and DPA, up to millimolar concentrations, present no toxicity at 24/48 h in HepG2 cells, as assessed by the CellTiter-Glo® Luminescent Cell Viability Assay (CTG) and the Neutral red uptake assay (NR) (N=4).

FIG. 9

(A) Control and WD rats housed in metabolic cages. WD rats were treated with D-PA and MB-OB3b. Body weight was followed in 24-hour periods. Body weight change was calculated relative to initial body weight MB-OB3b treated rats slightly loose body weight during a 4-days treatment period (N=4(+/−), N=4 (−/−), N=5 (DPA), N=4 (MB-OB3b) *p≤0.05 compared to untreated Atp7b−/− control)

(B) ARBM101 and MB-OB3b treatment induced a significant fecal copper excretion vs intake whereas DPA brought minor copper amounts into urine. Methanobactin-induced total copper excretion over 24 h exceeded the amount of ingested copper causing a negative copper balance. Total copper excretion over 24 h depicted as % of intake (N=3-4 (7 in case of ARBM101), *p≤0.05; **p≤0.01 compared to −/− control; #p≤0.05; ####p≤0.0001 compared to +/− control).

(C) DPA induced zinc excretion into urine and feces (in μmol/24 h) within 24 h of a 4-days treatment period (N=4 (7 in case of ARBM101), *p≤0.05; ****p≤0.0001 compared to −/− control; #p≤0.05 compared to +/− control).

FIG. 10

(A) Progressive liver copper accumulation in WD rats reaching a plateau around 90 days of age. At this age liver damage can be assessed by serum ALT and AST activity and by elevated bilirubin serum levels. In contrast to an early on continuous increase of hepatic copper, liver damage occurs rather abruptly around 90 days of age (N=2-38 (Atp7b+/−), N=11-63(Atp7b−/−) **p≤0.01, ****p≤p≤0.0001 compared to untreated Atp7b−/− control; #p≤0.05; ###p≤p≤0.001; ####p≤0.0001 compared to untreated Atp7b+/− control).

(B) Ceruloplasmin activity (CP) in serum samples of WD rats. Hardly any CP activity can be detected in still healthy (H) WD animals that nevertheless strongly increases in diseased WD rats (N=72 (+/−), N=45 (−/−H), N=36 (−/−D), ****p≤0.0001 compared to −/−H; ####p≤0.0001 compared to +/− control).

FIG. 11

Serum AST (green) and ALT (red) levels of four long-term ARBM101 treated WD rats. Treatment cycles are delimited by vertical dashed green lines and typically lasted for eight treatments days. Thresholds indicative for disease onset are given as dashed horizontal lines.

FIG. 12

Representative images of FFPE-HE stained liver tissue sections of age-adapted control rats, healthy (H) and WD rats with beginning hepatitis (Do), and 5 cycles ARBM101 long-term treated (LT) WD rats.

FIG. 13

(A) Electron micrographs of isolated liver mitochondria. Mitochondria from untreated diseased (D) WD rats demonstrate membrane detachments and dilated cristae. In contrast, liver mitochondria from long-term ARBM101 treated (LT) WD rats appeared structurally intact as the ones of control animals. Scale bars: upper panels 1000 nm, lower panels 500 nm.

(B) Electron micrographs of liver in-situ. Diseased (D) WD rats have prominent mitochondrial structure impairments in contrast to control animals and long-term ARBM101 treated (LT) WD rats Scale bars: upper panels 1000 nm, lower panels 500 nm.

FIG. 14

Chemical structures of full-length mbs from M. trichosporium OB3b (A) (144, 155), Methylocystis sp. M (B) (136), M. hirsuta CSC1 (C) (136), M. rosea (D) (136) and Methylocystis sp. SB2 (E) (135).

FIG. 15

Sequences detected in bacteria of known genome sequence from methanotrophs with structurally characterized mbs are shown in red, sequences detected in bacteria of known genome sequence from methanotrophs are shown in blue and sequences detected in bacteria of known genome sequence from non-methanotrophs are shown in green. Bar above amino acids represent the amino acid pair that is or proposed to be post-translationally modified into an oxazolone, imidazolone or pyrazinedione group. Abbreviations: methanobactin from Methylosinus trichosporium OB3b (mb-OB3b), Methylosinus sp. LW3 (mb-LW3), LW4 (mb-LW4), PW1 (mb-PW1), Methylocystis parvus OBBP (mb-OBBP), Methylocystis rosea (mb-rosea), Methylocystis sp. SB2 (mb-SB2), SC2 (mb-SC2), and LW5 (mb-LW5), Cupriavidus basiliensis B-8 (mb-B-8), Pseudomonas extremaustralis 14-3 (mb-14-3), Azospirillum sp. B510 (mb-B510), Tistrella mobilis KA081020-065 (mb-mobilis) and Comamonas composti DSM 21721 (mb-21721).

FIG. 16

Gene clusters of complete genomes of methanotrophs M. trichosporium OB3b, Methylocystis sp. SB2 and Methylocystis rosea.

### DETAILED DESCRIPTION

Wilson Disease (WD), an autosomal recessively inherited copper overload disorder, is a yet incurable disease that is fatal when left untreated. The overall therapeutic approach is the restoration and maintenance of normal copper homeostasis, either by medical therapy or by liver transplantation. Copper chelators (such as D-penicillamine, trientine and tetrathiomolybdate) and/or zinc salts presently represent the gold standard of WD treatment. Regardless of the specific approach chosen, treatment must be continued throughout the patient's lifetime, because abnormal copper accumulation cannot be controlled by a low copper diet. Importantly, non-adherence or discontinuation of medical therapy is associated with the risk of intractable hepatic or neurologic deterioration.

Presently available treatment options are, unfortunately, only of limited efficacy in terms of reducing copper levels; and are moreover incapable of restoring physiological copper excretion via the bile. Along with severe side effects, the need for high dosages and repeated administration (often several times a day), results in a severe impairment of quality of life and overall poor patient adherence.

Moreover, commonly prescribed WD therapeutics fail to restore liver function once WD manifests as advanced liver failure—e.g., due to delayed diagnosis, poor compliance, or rapid, fulminant hepatitis. In this case, liver transplantation with all its inherent risks and detriments presently remains the only viable option. Although liver transplantation is effective to restore normal biliary copper excretion (thereby preventing disease recurrence) and promotes removal of copper from extrahepatic sites, given the chronic shortage of suitable donor organs and the substantial morbidity and mortality associated with the procedure, it is considered as a treatment option only in life-threatening circumstances.

Novel means and methods for WD treatment are thus urgently needed. The surprising findings underlying the present invention show that methanobactins, methanotroph-derived chalkophores, are surprisingly potent and well-tolerated de-coppering agents.

Unexpectedly, the present inventors found that due to their superior copper binding affinity, methanobactins (in contrast to other copper chelators, being far less efficient) can advantageously be used for massive depletion of (excess) copper with a long-term effect—thereby allowing for a novel treatment regimen that is expected to markedly improve patient compliance and, consequently, overall therapeutic success. Moreover, the present inventors found that methanobactins are even capable of removing accumulated mitochondrial copper—which has recently been suggested as a crucial causative factor for oxidative stress underlying tissue and organ damage in a number of diseases. Therefore, methanobactins are not only promising agents for treatment of WD, but also for a number of unrelated diseases that have been linked to increased copper levels in the blood, in whole cells and/or in mitochondria within.

### Wilson Disease

Wilson Disease (WD) is an inherited disorder associated with mutations in the copper transporting ATPase ATP7B, resulting in impaired, non-functional or impaired ATP7B protein activity. More than 500 mutations in ATP7B have been identified, most of which are low-abundance mutations.

WD is typically characterized by severe impairment (or even complete absence) of biliary copper excretion, resulting in hepatic copper overload and, eventually, copper spillover into the circulation and/or central nervous system.

A variety of signs and symptoms reflecting cellular injury from excess copper may be present in affected patients. Many types of liver disease may be encountered in patients with Wilson Disease, and presenting symptoms of liver disease can be highly variable, ranging from asymptomatic, with only biochemical abnormalities, to overt cirrhosis. Wilson Disease may also present as acute liver failure as described elsewhere herein. Other manifestations include Coombs positive hemolytic anemia, cardiomyopathy, and endocrine dysfunction. Neurologic signs, more common in the second or third decade of life, are variable, and most often include tremor, ataxia, and dystonia, consistent with neuropathologic findings of basal ganglia involvement. The most common psychiatric features are abnormal behaviour (typically increased irritability or disinhibition), personality changes, anxiety, and depression.

Diagnosis of WD typically requires a combination of tests that are reflected by the diagnostic score that was proposed by the Working Party at the 8^{th} International Meeting on WD, Leipzig 2001 (Ferenci et al. Liver Int. 2003; 23(3):139-42) and is now included in the European Association for the Study of the Liver (EASL) clinical practice guidelines for Wilson Disease (EASL Clinical Practice Guidelines: Wilson's disease, J Hepatol. 2012 March; 56(3):671-85). Often, the combination of Kayser—Fleischer rings and a low serum ceruloplasmin decreased by 50% of the lower normal value, typically 0.1 g/L or less, is sufficient to establish a diagnosis. Kayser—Fleischer rings are caused by deposition of copper in Desgemet's membrane of the cornea and can be assessed by slit lamp examination. ATP7B loss-of-function and consequent failure to incorporate copper during ceruloplasmin biosynthesis results in the secretion of an apoprotein that is devoid of enzymatic activity and rapidly degraded, accounting for low serum concentrations of enzymatically active ceruloplasmin and thus proportionally low total serum concentrations of copper typically seen in WD patients, except in cases of severe liver injury or acute liver failure, when there are high serum concentrations of non-ceruloplasmin-bound copper due to its sudden release from the liver.

Other important diagnostic parameters according to the EASL Clinical Practice Guidelines (loc. cit.) include increased urinary copper excretion (>1.6 μmol/24 h or >0.64 μmol/24 h in children), non-ceruloplasmin-bound copper (“free copper”) levels >1.6 μmol/L and a hepatic parenchymal copper content of >4 μmol/g dry weight. Direct genetic testing for ATP7B mutations are also increasingly available to confirm clinical WD diagnosis.

Notably, methanobactin treatment according to the present invention is in general envisaged for WD manifesting by any of the aforementioned signs and symptoms. Due to their superior copper binding affinity, methanobactins are considered useful in any form of WD. Unless noted otherwise, the term “Wilson Disease” or “WD” thus includes acute and non-acute forms of WD, presenting with hepatic and/or neurological deficits, early onset WD in infancy and late-onset WD in adults, previously treated and untreated WD. Advantageously, methanobactins, particularly administered according to the treatment regimen provided herein, are also considered to be effective when otherwise liver transplantation would be indicated, including WD patients with acute liver failure as the first presentation of disease, non-responders to conventional copper chelator therapy, those who present with end-stage liver disease (ESLD) and severe hepatic insufficiency, and patients with neurological WD in the absence of liver failure as reviewed by Schilsky M L, Ann. N. Y. Acad. Sci. 2014 May; 1315:45-9. Also encompassed by the term are related copper-overload diseases in non-human mammalian subjects, including dogs. The term WD also includes animal models of WD, such LPP−/− rats carrying an ATP7B mutation that completely abolishes its hepatic copper transport activity.

In general, patients presenting with any of the manifestations mentioned in the foregoing are envisaged to benefit from methanobactin therapy. Particularly, (recurring) treatment cycles of massive copper depletion as a result of methanobactin administration according to the treatment regimen described herein are envisaged as an effective, well-tolerated and patient-compliant treatment option for WD presenting with any of the aforementioned signs and symptoms.

### Treatment Regimen

Accordingly, in a first aspect, the present invention provides a method of treating Wilson Disease in a subject, wherein the treatment comprises at least one treatment cycle of (a) a first phase of methanobactin administration followed by (b) a second phase of non-treatment, wherein the second phase exceeds the first phase. The methanobactin may be a Methylosinus trichosporium OB3b methanobactin (mb-OB3b) or a Methylocystis sp. SB2 methanobactin (ARBM101). “Non-treatment” refers to a period of time during which no methanobactin is administered. Optionally and advantageously, “non-treatment” may include that no other WD therapeutics (in particular copper chelators) are administered. Surprisingly, it turned out that methanobactins as described herein are extremely efficient and well-tolerated de-coppering agents that allow for (recurrent) treatment phases of massive copper depletion with a long-term effect. I.e., the present inventors discovered that steady administration (as with copper chelators known in the art) is not necessarily required when using methanobactins for WD treatment, but that patients can rather undergo (recurrent) phases of methanobactin treatment for removing excess copper, followed by phases that preferably do not require administration of WD therapeutics at all. This is a significant advantage over currently known WD therapeutics which often require life-long, steady administration in high dosages. The treatment regimen according to the present invention is therefore expected to markedly improve quality of life of WD patients, and, thereby, patient adherence and overall therapeutic success.

Particularly, the first phase of the inventive treatment regimen is envisaged to last for a period of 1, 2, 3, 4, 5, 6, 7, 8, 9, 10 or more consecutive days. During the first phase of treatment (“de-coppering phase”), the methanobactin may be administered as described elsewhere herein either in single doses once daily, twice daily, three times daily, four times daily, five times daily, every other day, or continuously. The first phase of administration of the methanobactin is followed by a second phase of non-treatment. Advantageously, said second phase is even thought to exceed the first phase of methanobactin administration as demonstrated in the appended examples, and is hence envisaged to last for at least about 1, 2, 3, 4, 5, 6, 7, 8, 9, 10, or more weeks or even longer. As will be readily acknowledged by the skilled practitioner, the duration of the second phase will depend on several factors, e.g. nutritional copper intake, body constitution, severity of WD, etc. Nevertheless, a minimum period of non-treatment of at least about 1 week is envisaged herein after the first phase of methanobactin administration.

It will be readily understood that recurrent treatment cycles are envisaged, i.e. several treatment cycles as described in the foregoing may follow one another. Specifically, a phase of non-treatment may be followed by a phase of treatment (de-coppering phase), and a subsequent phase of non-treatment may be followed by another de-coppering phase, and so on. Treatment cycles may be reiterated in intervals, over several weeks, months, years, or even life-long. The treatment regimen of the present invention provides for prophylactic depletion of copper on a regular basis (i.e. before signs and symptoms of WD occur) and/or for acute and optionally recurrent de-coppering treatment whenever necessary. The skilled practitioner will readily be able to assess when methanobactin treatment according to the invention is indicated.

### Acute WD

As explained previously, the present invention provides a novel and effective treatment regimen that allows for (optionally repeated) copper depletion in WD patients. Another surprising insight underlying the present invention is the fact that methanobactins are effective for treatment of acute WD presenting as acute liver failure (ALF); a condition that was, to date, invariably fatal unless liver transplantation was conducted.

Acute WD is defined herein as WD manifesting as acute liver failure (ALF), which may be the initial presentation of WD or can occur when WD treatment is stopped. Known copper chelators presently used for WD therapy are, by far, not able to bind to and remove enough excess copper to remedy the rapid deterioration of liver function seen in WD patients presenting with ALF. In contrast, methanobactins as described herein have surprisingly been found to be capable of depleting copper so efficiently that even WD patients presenting with acute WD—manifesting as ALF—are envisaged to be effectively treatable without the need of emergency liver transplantation.

Acute liver failure is defined as the rapid development of hepatocellular dysfunction (i.e. within less than 26 weeks from the onset of the first hepatic symptoms), optionally accompanied by coagulopathy and hepatic encephalopathy in a patient. Hepatic encephalopathy may present as deficits in higher brain function (e.g. mood, concentration in grade I) to deep coma (grade IV). Coagulopathy typically manifests as a prolongation in prothrombin time (usually an International Normalized Ratio (INR) ≥1.5), and progressive thrombocytopenia (detectable in a full blood count).

Diagnosis of ALF is based on physical exam, laboratory findings and patient history. On laboratory testing, liver function can be assessed by evaluating aspartate transaminase (AST), alanine transaminase (ALT), alkaline phosphatase (ALP), gamma glutamyl transpeptidase (GGT), total bilirubin and/or albumin levels. Subjects with ALF due to WD often present with a nonimmune (Coombs-negative) hemolytic anemia that may precede the development of liver failure or occur concurrently with the liver injury. Decay of liver cells may result in the release of large amounts of stored copper into the circulation, thereby increasing “free” (non-ceruloplasmin bound) copper levels. An increase in the alkaline phosphatase (ALP) to bilirubin ratio of less than 4:1 owing to the relative decrease in alkaline phosphatase (ALP) and increased bilirubin (resulting from hemolysis and hepatic dysfunction), a ratio of aspartate transaminase (AST) to alanine transaminase (ALT) of greater than 2.2:1, and increases in serum copper, typically above 200 μg/dL, are suggestive of ALF due to acute WD. Guidance as to how to identify such patients is i.a. provided by Schilsky M L, Ann. NY Acad. Sci. 2014 May; 1315:45-9 and Bermann et al. Gastroenterology. 1991 April; 100(4):1129-34. Particularly, if both the alkaline phosphatase (ALP) to bilirubin ratio is greater than 4:1 and the AST to ALT ratio is above 2.2 concurrently, ALF due to WD can be assumed.

Diagnosis can be confirmed by evaluating other signs and symptoms suggesting Wilson Disease, including clinical symptoms (e.g. deep jaundice) and the conventional WD diagnostic parameters (ceruloplasmin, serum or urinary copper as described elsewhere herein). The diagnosis has to be ascertained by determining the hepatic copper content by liver biopsy and/or mutation analysis as described previously.

The clinical presentation of acute WD typically progresses rapidly from hepatic to renal failure and, when untreated, leads to almost 95% mortality unless emergency liver transplantation is available. The present inventors were the first to acknowledge that methanobactins can be used as an effective remedy of the severe clinical manifestations of acute WD. It is contemplated that copper depletion by administration of methanobactins may even render liver transplantation in acute WD patients obsolete. Methanobactin treatment of acute WD can be carried out according to the treatment regimen described elsewhere herein, or according to any other treatment scheme that the skilled practitioner considers appropriate. Typically, acute WD treatment will involve a phase of massive copper depletion by administration of a sufficient amount of methanobactin, that may be ended once the signs and symptoms of acute WD subside; and/or laboratory values improve. Subsequently, treatment according to the regimen of the present invention may follow.

### Methanobactin

As set out elsewhere herein, the present inventors were the first to acknowledge the therapeutic potential of methanobactins as safe and effective copper depleting agents for WD treatment according to a novel treatment regimen, and for treatment of acute WD that was to date considered to be irreversible by drug therapy. The term “methanobactin” or “mb” as used herein generally refers to a copper-binding (and Cu(II)-reducing) peptide derived from bacteria, particularly methanotroph bacteria. Unless denoted otherwise, “copper” is used herein to refer to both Cu(I) and Cu(II). Naturally occurring methanobactins are thought to be secreted to the extracellular media where they function as chalkophores by binding to Cu(II) or Cu(I) and shuttling the copper into the cell.

The term “methanobactin” as used herein in particular encompasses modified peptides characterized by the presence of one oxazolone ring and a second oxazolone, imidazolone or pyrazinedione ring. The two rings are separated by 2-5 amino acid residues. Each ring has an adjacent thioamide group. Structurally, mbs can be divided into two groups that are both envisaged for the uses according to the present invention (FIGS. 11, 12). One type (Group I) is represented by mb from Methylosinus trichosporium OB3b. Based on sequence similarity and alignments, the putative mbs from Methylosinus sp. LW3 (mb-LW3), Methylosinus sp. LW4 (mb-LW4), Methylosinus sp. PW1 (mb-PW1), Methylocystis sp. LW5 (mb-LW5) and one of the two mbs from Methylocystis parvus OBBP (mb-OBBP(2)) would also fall within this group (FIG. 15). In this group the rings are separated by 4 or 5 amino acids and the mb contains 2 or more Cys not involved in ring formation.

The second group (group II) is represented by the structurally characterized mbs from Methylocystis sp. S82, rosea and SC2 (FIGS. 14, 15). This mb group lack the Cys in the core peptide, are smaller and probably less rigid, due to the absence of the disulfide bond found in mb-OB3b. In this group the rings are separated by two amino acids. In contrast to the other members of group II mbs, mb-B-8, mb-14-3, mb-B510 and mb-21721 contain 4 Cys. However, based on the location of the Cys we predict all 4 Cys are modified into the heterocyclic rings. Mbs from the structurally characterized members in this group contain a sulfate group, which may aid in the formation of a tight bend by making a hydrogen bond with the backbone amide of Ser2. The sulfate group also increases Cu^{2+/1+} affinity (El Ghazouani et al., 2012. Proc. Nat. Acad.Sc. 109: 8400). The conserved T/S adjacent to the C-terminal ring suggests that the other members of this group also contain a sulfate group.

It was discovered that the genome region of the putative mb precursor matching sequence in M. trichosporium OB3b had a number of distinctive and striking features (FIG. 16). These include (a) a precursor peptide translationally modified peptide; (b) a potential cleavage site between the leader and core peptide, suggestive of secretion; (c) genes upstream and downstream of the mb gene cluster encoding protein sequences compatible with possible roles in maturation of the mb precursor sequence, transport, and regulation of mb biosynthesis. Elaboration on this initial search revealed a series of genomes containing gene clusters with characteristics matching that of the M. trichosporium OB3b mb gene cluster, e.g. in Methylocystis parvus OBBP, Methylosinus sp. LW3 as well as non-methanotrophs Azospirillum sp. B510, Azospirillum sp. B506, Pseudomonas extremaustralis Pseudomonas extremaustralis substrain laumondii TT01 Tistrella mobilis, Gluconacetobacter sp. SXCC. Gluconacetobacter oboediens Methylobacterium sp. B34 , Cupriavidus basilensis B-8, Photorhabdus luminescens and Vibrio caribbenthicus BAA-2122.

At present the only genes in the Methylosinus trichosporium OB3b mb gene cluster with a known function are the structural gene for mb-OB3b, MbnA, and TonB-transporter (MbnT) which is responsible for Cu⁺-mb-OB3b uptake (Semrau et al., unpublished results). The cytochrome c peroxidase MbnH, and the FAD⁺-dependent oxidoreductase, present instead or sometimes in addition to MbnH in methanotroph gene clusters MbnF are likely candidates to be involved in the oxidation steps required for ring formation. In addition, the aminotransferase MbnN found in the mb-OB3b, but not the mb-SB2 gene cluster may be involved in formation of the N-terminal keto-isopropyl group, and the sulfotransferase MbnS found in the mb-SB2 and mb-rosea, but not the mb-OB3b gene cluster may catalyse sulfonation of the threonine. One other gene product, the multidrug and toxin extrusion (MATE) protein has been suggested to be involved in secretion of mature mbs.

Generally, the present invention encompasses methanobactins encoded by a mb gene, preferably a Methylosinus trichosporium OB3b mb gene or variants or orthologs thereof, or a Methylocystis sp. SB2 mb gene or variants or orthologs thereof. The term “variant” in reference to a nucleic acid sequence refers to polymorphisms, i.e. the exchange, deletion, or insertion of one or more nucleotides, respectively, as compared to the “parent” nucleic acid sequence that the variant is derived from. “Orthologs”, or orthologous genes, are genes in different species that evolved from a common ancestral gene by speciation. As used herein a variant or ortholog encodes a copper-binding methanobactin preferably exhibiting the same advantageous properties as the mb evaluated in the appended examples. It is envisaged that the variant or ortholog of the mb-OB3b gene or the variant or ortholog of the mb-SB2 gene comprises or consists of a nucleic acid sequence having at least about 60%, such as at least about 65%, 70%, 75%, 80%, 85%, 90%, 95% or 99% sequence identity to the mb gene.

The mb-OB3b gene, variant or ortholog thereof is envisaged to encode an mb precursor peptide that comprises or consists of an amino acid sequence that has at least about 60%, such as at least about 65%, 70%, 75%, 80%, 85%, 90%, 95% or 99% sequence identity to the amino acid sequence of the known mb-OB3b precursor peptide with UniProt Acc. No. E3YBA4 (entry version No. 15 of Jun. 24, 2015) and as depicted in SEQ ID No. 1 (FIG. 15). Particularly, and as described in more detail below, the mb OB3b gene was found to encode a precursor peptide including a leader peptide and a core peptide, separated by a potential cleavage site. Preferred % sequence identities for the overall precursor peptide are indicated above. Moreover, the encoded (i.e., non-translationally modified) methanobactin (i.e., core peptide) is envisaged to comprise or consist of an amino acid sequence that has at least about 60%, such as at least about 65%, 70%, 75%, 80%, 85%, 90%, 95% or 99% sequence identity to the amino acid sequence of the known mb-OB3b core precursor peptide as depicted in SEQ ID No. 1 (FIG. 15).

The mb-SB2 gene, which is composed of the mbn operon, variant or ortholog thereof is envisaged to encode an mb precursor peptide that comprises or consists of an amino acid sequence that has at least about 60%, such as at least about 65%, 70%, 75%, 80%, 85%, 90%, 95% or 99% sequence identity to the amino acid sequence of the known mb-SB2 precursor peptide as depicted in SEQ ID No. 7 (FIG. 15). Particularly, and as described in more detail below, the mb SB2 gene was found to encode a precursor peptide including a leader peptide and a core peptide, separated by a potential cleavage site. Preferred % sequence identities for the overall precursor peptide are indicated above. Moreover, the encoded (i.e., non-translationally modified) methanobactin (i.e., core peptide) is envisaged to comprise or consist of an amino acid sequence that has at least about 60%, such as at least about 65%, 70%, 75%, 80%, 85%, 90%, 95% or 99% sequence identity to the amino acid sequence of the known mb-SB2 core precursor peptide as depicted in SEQ ID No. 7 (FIG. 15).

Generally, the term “sequence identity” indicates the extent to which two (nucleotide or amino acid) sequences have identical residues at the same positions in an alignment, and is often expressed as a percentage. Preferably, identity is determined over the entire length of the sequences being compared. Thus, two copies of exactly the same sequence have 100% identity, but sequences that are less highly conserved, and have deletions, additions, or replacements, may have a lower degree of identity. Those skilled in the art will recognize that several algorithms are available for determining sequence identity using standard parameters, for example Blast (Altschul, et al. (1997) Nucleic Acids Res. 25:3389-3402), Blast2 (Altschul, et al. (1990) J. Mol. Biol. 215:403-410), Smith-Waterman (Smith, et al. (1981) J. Mol. Biol. 147:195-197) and ClustalW. Accordingly, for instance, the amino acid sequences of SEQ ID No: 1 may serve as “subject sequence” or “reference sequence”, while the amino acid sequence or nucleic acid sequence of a polypeptide or polynucleotide different therefrom can serve as “query sequence”.

A high affinity for copper is a common feature of methanobactins. Therefore, methanobactins of the invention are envisaged to bind copper—specifically Cu(I)—with high binding affinity. The term “affinity” or “binding affinity” refers to the strength of the binding of a ligand, such as a methanobactin to Cu(I). The affinity of the binding of a given ligand to its target is often determined by measurement of the on-rate constant (kₒₙ) and off-rate constant (k_{off}) and calculating the quotient of k_{off} to kₒₙ to obtain the equilibrium dissociation constant K_{d}(K_{d}=k_{off}/kₒₙ) which is inversely related to the binding affinity, i.e. the lower the K_{d} value, the higher the binding affinity. Preferred methanobactins of the invention bind Cu(I) with an equilibrium dissociation constant or K_{d} in the nanomolar range, i.e. 10⁻⁷, 10⁻⁸, 10⁻⁹, in the picomolar range, i.e. 10⁻¹⁰, 10⁻¹¹, 10⁻¹², or in the femtomolar range, i.e. 10⁻¹³, 10⁻¹⁴, 10⁻¹⁵. Preferably, methanobactins of the invention bind Cu(I) with a K_{d} in the femtomolar range, and are particularly envisaged to bind Cu(I) with a K_{d} of 10⁻¹⁵ or less. A number of different methods have been used to determine metal binding affinity constants for mbs. All measurement methods show Cu(II)/(I) and Cu(I) affinities of ˜10²¹ M⁻¹ or greater, and is one of the highest known for biological systems. With respect to this proposal, mb-OB3b has been shown to remove Cu from metallothionein in both in vitro and in vivo experiments. Mbs have been shown to solubilize and bind insoluble forms of Cu(I) under anaerobic conditions, and to extract Cu from copper minerals, humic materials, and glass Copper (Cu(I)) binding affinity can for example be measured according the ESI-MS approach of Banci et al. (Nature. 2010 Jun. 3; 465(7298):645-8) which relies on the simultaneous monitoring of the variation in the metallated/non-metallated Cu(I) binding ligand ratios at increasing concentrations of a competing ligand, namely dithiothreitol (DTT) or diethyl-dithio-carbamate (DETC). Alternatively, Cu(I) binding affinities can for example be determined from competition titrations with the chromophoric copper chelator bathocuproine disulfonate (BCS) as described by El Ghazoiani A et al., Proc. Natl. Acad. ScL USA. 2012 May 29; 109(22):8400-4. Measuring Cu(I) binding affinities with this method, methanobactins encompassed by the present invention will also exhibit a K_{d} of 10⁻¹⁵ or less, such as 10⁻¹⁶, 10⁻¹⁷, 10⁻¹⁸, 10⁻¹⁹, 10⁻²⁰, 10⁻²¹ or less.

As set out previously herein, methanobactins exhibiting high copper binding affinities (and binding Cu(I) with a K_{d} of 10⁻¹⁵ or less, “high-affinity mb”) are particularly envisaged for the uses according to the present invention, and in particular for massive copper depletion in (acute) WD therapy. However, methanobactins with a higher Ka (i.e. binding Cu(I) with a lower affinity) can also be successfully employed in treatment of a variety of diseases. E.g., in cases when a less extensive and/or quick copper depletion is desired, methanobactins with a lower binding affinity towards Cu(I) (“low-affinity mb”) can be utilized. It is also contemplated to combine methanobactins with different Cu(I) binding affinities for treatment. E.g., one or more treatment cycles with a high-affinity mb for extensive removal of copper from a patient can be followed by one or more treatment cycles with a low-affinity mb for maintenance therapy in order to keep copper levels low without excessively depleting copper. Vice versa, treatment could also be started with low-affinity mb and, after one or more treatment cycles, optionally gradually be continued using mbs with a higher Cu(I) binding affinity.

The term “methanobactin” includes naturally occurring methanobactins and functional variants, fragments and derivatives thereof which retain the capability of complexing copper (i.e., Cu(I) and Cu(II)), and preferably bind Cu(I) with a binding affinity that is comparable or even higher than that of the naturally occurring methanobactins.

As set forth previously, the methanobactin of the invention may be derived from bacteria listed in FIG. 15, including Methylocystis spec., Methylosinus spec., Methylomicrobium spec. and Methylococcus spec. Particularly, the methanobactin may be selected from (a) a Methylosinus trichosporium OB3b methanobactin (mb-OB3b) (b) a Methylocystis sp. SB2 methanobactin (mb-SB2), (c) a Methylococcus capsulatus Bath methanobactin (mb-Bath) (d) a Methylomicrobium album BG8 methanobactin (mb-BG8), (e) a Methylocystis sp. M methanobactin, (f) a Methylocystis hirsuta CSC1 methanobactin and (g) a Methylocystis rosea methanobactin (mb-rosea), (h) a Methylosinus sp. LW3 methanobactin (mb-LW3), (i) a Methylosinus sp. LW4 methanobactin (mb-LW4), (j) a Methylocystis sp. LW5 (mb-LW5), (k) a Methylosinus sp. PW1 methanobactin (mb-PW1), (I) a Methylocystis parvus OBBP methanobactin (mb-OBBP), (m) a Cupriavidus basiliensis B-8 methanobactin (mb-B-8), (n) a Pseudomonas extremaustralis 14-3 methanobactin (mb-14-3), (o) a Azospirillum sp. B510 methanobactin (mb-B510), (p) a Tistrella mobilis KA081020-065 (mb-mobilis) methanobactin and (q) a Comamonas composti DSM 21721 methanobactin (mb-21721).

Methanobactins selected for the uses according to the present invention preferably have the same advantageous properties as the mb evaluated in the appended examples and/or as described elsewhere herein.

In general, methanobactin of the invention may comprise, or consist of, the following general formula (I):

R¹—(X)₂₋₅—R² (I)

wherein
R¹ and R² are each a 5-membered heterocycle comprising N and associated with an enethiolate;
and each X is independently selected from any amino acid.

The term “amino acid” or “amino acid residue” typically refers to an amino acid having its recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (Ile or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Ile, Leu, Met, Gly, Phe, Pro, Val); a negatively charged side chain (e.g., Asp, Glu); a positively charged sidechain (e.g., Arg, His, Lys); or an uncharged polar side chain (e.g., Asn, Cys, Gln, Ser, Thr, Trp, and Tyr). The term encompasses naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Particularly, where the methanobactin is mb-OB3b, it is contemplated to comprise or consist of the formula (II)

R¹GSCYR²SCM (II),

wherein R¹ is selected from (N-2-isopropylester(4-thionyl-5-hydroxy-imidazole) and N-2-isopropylester-(4-thiocarbonyl-5-hydroxy-imidazolate), and R² is selected from pyrrolidine—(4-hydroxy-5-thionyl-imidazole) and pyrrolidine-(4-hydroxy-5-thiocarbonyl-imidazolate). Said mb-OB3b may in particular comprise or consist of the formula (IV):

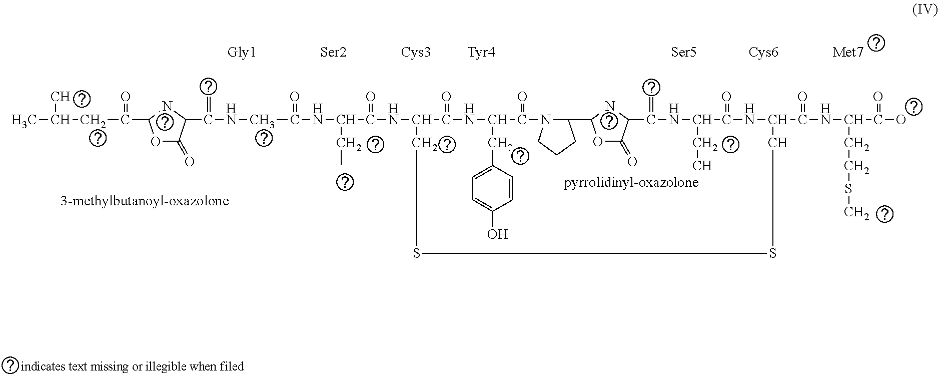 [SMILES: C=C(NCC(=O)NC(CC)C(=O)NC1CSSCC(C(=O)NC(CCSC)C(=O)O)NC(=O)C(CC)NC(=C)C2NC(OC2=O)C2CCCN2C(=O)C(Cc2ccc(O)cc2)NC1=O)C1NC(C(=O)CC(C)C)OC1=O]

When complexing zinc or copper, said mb-OB3b is envisaged to have the following structure (VI)

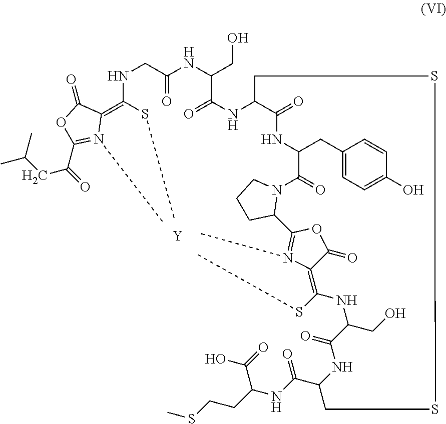

wherein Y is selected from copper (Cu(I) or Cu(II)) or zinc (Zn(I) or Zn(II)).

Where the methanobactin is mb-SB2, it is envisaged to be of the formula (III)

R¹ASR²AA (III)

wherein R¹ is 4-guanidinobutanoyl-imidazole and R² is 1-amino-2-hydroxy- oxazolone.

Said mb-SB2 may in particular be of the formula (V):

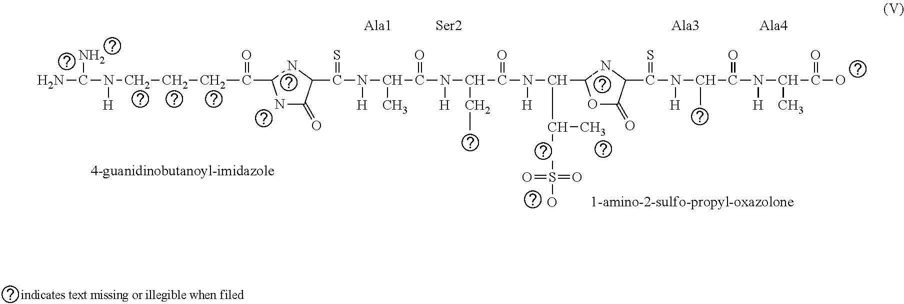 [SMILES: CCC(NC(=O)C(C)NC(=S)C1NC(C(=O)CCCNC(N)N)NC1=O)C(=O)NC(C(C)CS(=O)(=O)O)C1NC(C(=S)NC(C)C(=O)NC(C)C(=O)O)C(=O)O1]

When complexing zinc or copper, said mb-SB2 is envisaged to have the following structure (VII):

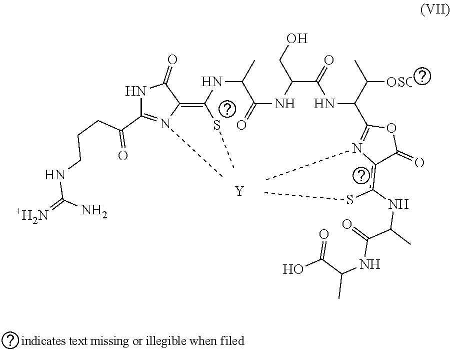

wherein Y is selected from copper (Cu(I) or Cu(II)) or zinc (Zn(I) or Zn(II).

This specific MB peptide mb-SB2 from Methylocystis sp. SB2 acts most effectively as a promising copper chelator compared to existing clinically approved cooper chelators such as D-PA. mb-SB2 even depletes copper at least as effective as another MB peptide mb-OB3b derived from Methylosinus trichosporium OB3b, as mentioned above.

MB peptide mb-SB2 from Methylocystis sp. SB2 is structurally and chemically deviating from other MB peptides, especially from mb-OB3b derived from Methylosinus trichosporium OB3b. Yet, a structurally different and less heavy peptide within the MB-family exhibits a similar therapeutic potential as a copper chelator.

When used herein, the terms “complexing” and “binding” are used interchangeably, i.e. for instance a methanobactin “binding” copper is to be understood as a methanobactin “complexing” copper, and vice versa. The term “complexing” generally means forming a complex consisting of a central ion and surrounding array of molecules that are known as ligands or complexing agents. For the present invention, the central ion will be copper (i.e. Cu(I) or Cu(II)), or zinc (i.e. Zn(I) or Zn(II)) and the ligand will be methanobactin. One methanobactin will typically complex one copper or zinc ion, forming a methanobactin-copper complex or a methanobactin-zinc complex, respectively. The person skilled in the art will readily understand that methanobactin-copper complexes will typically form after administration of the methanobactin to the subject, when methanobactin complexes and thereby depletes (excess) copper in the subject's body. Methanobactin-zinc complexes are envisaged herein as stabilized forms of methanobactin as described below.

As set out elsewhere herein, methanobactin fragments, variants and derivatives are also envisioned for the uses described herein.

“Methanobactin fragments” are “functional” or “copper-binding” peptides that retain the copper-binding region of the “parent” methanobactin they are derived from. It is for instance envisaged to provide particularly small methanobactin fragments that are capable of crossing the blood-brain-barrier in order to effectively treat neurological WD or other conditions associated with copper overload in the CNS.

The term “methanobactin variant” refers to methanobactins having the general methanobactin formula of a “parent” methanobactin (FIG. 15), but containing at least one amino acid substitution, deletion, or insertion as compared to the parent methanobactin, provided that the variant retains the desired copper-binding affinity and/or biological activities described herein.

“Methanobactin derivatives” are chemically modified methanobactins. Generally, all kind of modifications are comprised by the present invention as long as they do not abolish the beneficial effects of the methanobactins. That is, methanobactin derivatives preferably retain the copper-binding affinity and/or biological activity of the methanobactins they are derived from. Methanobactin derivatives also include stabilized methanobactins as described in the following.

Possible chemical modifications in the context of the present invention include acylation, acetylation or amidation of the amino acid residues. Other suitable modifications include, e.g., extension of an amino group with polymer chains of varying length (e.g., XTEN technology or PASylation®), N-glycosylation, O-glycosylation, and chemical conjugation of carbohydrates, such as hydroxyethyl starch (e.g., HESylation®) or polysialic acid (e.g., PolyXen® technology). Chemical modifications such as alkylation (e. g., methylation, propylation, butylation), arylation, and etherification may be possible and are also envisaged. Further chemical modifications envisaged herein are ubiquitination, conjugation to therapeutic or diagnostic agents, labeling (e.g., with radionuclides or various enzymes), and insertion or substitution by chemical synthesis of non-natural amino acids.

Other possible modifications may involve replacement of oxazolone group with the more stable imidazolone or pyrazinedione group. Gene additions and/or deletions of genes from the operons of Group II methanobactins into Group I or vice versa should result in alteration may result in a change in the type of ring. Replacement of oxazolone group(s) with either imidazolone or pyrazinedione group(s) should increase the stability of methanobactin to the point where oral administration may be possible.

For the purpose of the invention the methanobactin as defined above also includes the pharmaceutically acceptable salt(s) thereof. The phrase “pharmaceutically acceptable salt(s)”, as used herein, means those salts of methanobactins that are safe and effective for treatment. Pharmaceutically acceptable salts include those formed with anions such as those derived from hydrochloric, phosphoric, acetic, oxalic, tartaric acids, choline etc., and those formed with cations such as those derived from sodium, potassium, ammonium, calcium, ferric hydroxides, isopropylamine, triethylamine, 2-ethylamino ethanol, histidine, procaine, etc.

As set forth previously, the methanobactin fragments, variants and derivatives preferably retain the advantageous capabilities of the methanobactins evaluated in the appended examples.

### Biological Effect

As explained previously, methanobactins according to the present invention are envisaged to elicit the desired biological effects as described herein, e.g. they are preferably capable of binding copper with a high binding affinity, and effecting its depletion from the system and preferably its excretion via the bile. Without wishing to be bound by specific theory, the present inventors have established that mitochondrial impairment due to an increased copper load progressively increases with disease state in livers from LPP −/− rats, a model of WD. As shown in the appended examples, methanobactins are able to rapidly deplete mitochondrial and hepatocellular copper. The present inventors have further demonstrated that the enormous liver copper depletion achieved by methanobactins brought liver copper very close to physiological levels and allowed for a new treatment regimen consisting of intense treatment cycles followed by prolonged drug holidays that ensured a healthy animal life. It is methanobactins unique combination of immense copper affinity, inertness of bound copper, and sustained mobilization that results in an unprecedented net copper excretion. Within 24 hours, the most elevated levels of fecal excretion were quantified upon ARBM-101 treatment among those copper chelators including mb-OB3b. Importantly, no compound related toxicities were observed, neither in vitro nor in vivo, and no signs of immunological complications were observed in the liver tissues from the long-term treated animals. Treatments caused fully restored mitochondrial power and constant animal weight gain. It is envisaged that methanobactins as described herein preferably exhibit the same advantageous characteristics.

In yet another embodiment, methanobactins are envisaged for the use of treatment of Wilson Disease, wherein the treatment reduces (i) whole liver copper levels, (ii) overall hepatocyte copper levels and/or (iii) hepatocyte mitochondrial copper levels. Moreover, methanobactins will preferably effect excretion of (excess) copper via the bile.

### Therapeutic Effect

Administration of methanobactins to subjects in need thereof (in particular WD patients) is expected to elicit a therapeutic effect. The term “therapeutic effect” as used herein generally refers to a desirable or beneficial impact of a treatment, e.g. amelioration or remission of the disease manifestations. The term “manifestation” of a disease is used herein to describe its perceptible expression, and includes both clinical manifestations, hereinafter defined as indications of the disease that may be detected during a physical examination and/or that are perceptible by the patient (i.e., symptoms), and pathological manifestations, meaning expressions of the disease on the cellular and molecular level. Amelioration or remission of WD manifestations can be assessed by using the same tests as described for diagnosis of WD. Additionally or alternatively it is also possible to evaluate the general appearance of the respective patient (e.g., fitness, well-being) which will also aid the skilled practitioner to evaluate whether a therapeutic effect has been elicited. The skilled person is aware of numerous other ways which are suitable to observe a therapeutic effect of the compounds of the present invention.

### Stabilized Methanobactin

In a further aspect, the present inventors have discovered ways to provide methanobactins in stabilized form.

Without wishing to be bound by specific theory, it was discovered that mb-OB3b is susceptible to time- and/or temperature-dependent decay. Thus, in order to allow an increased biological half-life and/or plasma concentration of methanobactins in the subject's body during (and after) treatment, and therefore preferably improve therapeutic efficacy and provide for a long-term effect of methanobactin treatment, it is envisaged to provide stabilized forms of methanobactin. Generally, any form of chemical modification is conceivable that enables stabilization of the methanobactins (see also methanobactin derivatives). Specifically, the present invention provides stabilized forms of methanobactins complexing zinc, i.e. Zn(I) or Zn(II). Unless denoted otherwise, the term “zinc” generally refers to Zn(I) and/or Zn(II). Furthermore, the present inventors found that methanobactins can be stabilized when provided at a pH of ≥9. Thus, it is envisaged to provide stabilized forms of methanobactins, i.e. methanobactins complexing Zn(I) or Zn(II) and/or being provided at a pH of 9, 10, or 11, for the uses and methods of the present invention. In particular, such stabilized forms of methanobactins can be used for treatment of WD according to the treatment regimen set out elsewhere herein, and/or for treatment of acute phase WD. Stabilized forms of methanobactins as described herein have not been used as medication before. The present invention thus also comprises a pharmaceutical composition comprising a stabilized methanobactin, wherein said methanobactin complexes Zn(I) and/or Zn(II) and/or is provided at a pH≥9. The skilled practitioner will readily understand that when the methanobactin is provided at a pH≥9 for reasons of stabilization, the pharmaceutical composition comprising methanobactin (optionally complexing zinc) is required to have a pH≥9, too.

A pharmaceutical composition comprising a methanobactin complexing Zn(I) or Zn(II) can be provided by contacting an amount of Zn(I) and/or Zn(II) and an amount of methanobactin in a ratio of 1:1 in aqueous solution. Use of equimolar amounts of zinc and methanobactin may be beneficial in order to avoid an excess amount of free zinc ions in the pharmaceutical composition.

### Pharmaceutical Composition

As set out in the foregoing, a pharmaceutical composition comprising methanobactin, in particular in stabilized form, is also envisaged herein. In particular, said pharmaceutical composition is envisaged for the use of treatment of Wilson Disease, wherein the treatment reduces (i) whole liver copper levels, (ii) overall hepatocyte copper levels and/or (iii) hepatocyte mitochondrial copper levels. I.e., the pharmaceutical composition preferably comprises methanobactins complexing Zn(I) or Zn(II) and/or are provided in a pH≥9. Said composition may be stable at 37° C. for at least 20, 50, 75, 100, 125, 150 hours or more. Accordingly, further aspects of the invention include a pharmaceutical composition comprising (in particular, stabilized) methanobactin as described herein and the use of the said (stabilized) methanobactin for the manufacture of a pharmaceutical composition. The term “pharmaceutical composition” particularly refers to a composition suitable for administering to a human. However, compositions suitable for administration to non-human animals are also envisaged herein.

The pharmaceutical composition and its components (i.e. active ingredients and optionally excipients or carriers) are preferably pharmaceutically acceptable, i.e. capable of eliciting the desired therapeutic effect without causing any undesirable local or systemic effects in the recipient. Pharmaceutically acceptable compositions of the invention may in particular be sterile and/or pharmaceutically inert. Specifically, the term “pharmaceutically acceptable” may mean approved by a regulatory agency or other generally recognized pharmacopoeia for use in animals, and more particularly in humans.

The (stabilized) methanobactin described herein is preferably present in the pharmaceutical composition in a therapeutically effective amount. By “therapeutically effective amount” is meant an amount of methanobactin that elicits the desired therapeutic effect. The exact amount dose will depend on the purpose of the treatment, and will be ascertainable by one skilled in the art using known techniques. Therapeutic efficacy and toxicity can be determined by standard pharmaceutical procedures in cell cultures or experimental animals, e.g., ED₅₀ (the dose therapeutically effective to 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions that exhibit large therapeutic indices are generally preferred.

The pharmaceutical composition is envisaged to comprise a methanobactin as described herein, particularly in stabilized form, and preferably in a therapeutically effective amount, optionally together with one or more carriers, excipients and/or additional active agents.

“Excipients” include fillers, binders, disintegrants, coatings, sorbents, antiadherents, glidants, preservatives, antioxidants, flavoring, coloring, sweeting agents, solvents, co-solvents, buffering agents, chelating agents, viscosity imparting agents, surface active agents, diluents, humectants, carriers, diluents, preservatives, emulsifiers, stabilizers and tonicity modifiers. Exemplary suitable carriers for use in the pharmaceutical composition of the invention include saline, buffered saline, dextrose, and water.

### Additional Active Agents

The pharmaceutical composition may also comprise further active agents effective for treatment of the particular disease concerned. By way of example, active agents presently used for treatment of WD include the copper chelators d-penicillamine (D-PA), trientine (TETA) and tetrathiomolybdate (TTM), as well as zinc salts. For treatment of cancer, useful additional active agents include known chemotherapeutic agents, including alkylating agents, antimetabolites, anti-microtubule agents, topoisomerase inhibitors; cytotoxic antibiotics, and monoclonal antibodies. Active agents for treatment of neurodegenerative disorders include, without limitation, levodopa and derivatives thereof, dopamine agonists, MAO-B inhibitors, catechol-O-methyltransferase (COMT) inhibitors, anticholinergics, amantadine, cholinesterase inhibitors, memantine and riluzole. It is within the knowledge of the skilled person to choose suitable additional agents for treatment of a specific disease.

### Formulation

The pharmaceutical compositions of the invention can be formulated in various forms, e.g. in solid, liquid, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in a form which is particularly suitable for the desired method of administration. Processes known per se for producing medicaments are indicated in Forth, Henschler, Rummel (1996) Allgemeine und spezielle Pharmakologie und Toxikologie, Urban & Fischer.

### Administration

A variety of routes are conceivable for administration of the methanobactins and pharmaceutical compositions according to the present invention. Typically, administration will be accomplished parentally, but oral administration is also envisaged. Methods of parenteral delivery include topical, intra-arterial, intramuscular, subcutaneous, intramedullary, intrathecal, intraventricular, intravenous, intraperitoneal, intrauterine, intravaginal, sublingual or intranasal administration.

### Cancer Treatment

The methanobactins and pharmaceutical composition disclosed herein is also envisaged for treatment of various cancers. Many cancer types exhibit increased intratumoral copper and/or altered systemic copper distribution. It has been acknowledged that copper serves as a limiting factor for multiple aspects of tumor progression, including growth, angiogenesis and metastasis. Methanobactins and pharmaceutical compositions described herein are thus promising tools to inhibit these processes.

As reviewed by Denoyer et al., Metallomics. 2015 Nov. 4;7(11):1459-76, high serum copper concentrations are reportedly associated with a variety of cancers including lymphoma, reticulum cell sarcoma, bronchogenic and laryngeal squamous cell carcinomas, cervical, breast, stomach and lung cancers, and elevated serum copper has been found to correlate with the stage of the disease and its progression in colorectal and breast cancers as well as hematological malignancies, including chronic lymphoid leukemia, non-Hodgkin's lymphoma, multiple myeloma and Hodgkin's lymphoma. Elevated copper in malignant tissues has also been established in a range of cancer types, including breast, ovarian, cervical, lung, stomach and leukemia. The role of copper in cancer development and progression remains to be elucidated. Elevated levels of redox active copper may lead to oxidative stress and chronic inflammation which are intrinsically linked to malignant transformation of cells. Therefore, it has been proposed that elevated copper in tissues or serum may be a risk factor for carcinogenesis. Methanobactins and pharmaceutical compositions described herein could be used to reduce overall copper levels and thereby minimize the risk for developing cancer.

Copper has also been reported to influence various molecular pathways inducing a pro-angiogenic response. Copper is capable of directly binding to angiogenic growth factors, and to influence their secretion and expression via activation of NFκB. Moreover, copper has been found to directly influence the ability of cancerous cells to invade and metastasize.

Papa et al., Genes Cancer. 2014 April; 5(1-2):15-21 further reported that the copper-dependent dismutase SOD1 is overexpressed in many cancers to cope with elevated levels of reactive oxygen species (ROS) caused by deregulation of the anti-oxidant machinery of the mitochondrial matrix. Depletion of copper is envisioned to reduce overall SOD1 activity, and thereby diminishing tumor cell proliferation and survival. In accordance, methanobactins and pharmaceutical compositions described herein are also envisaged for treatment of cancers which overexpress SOD1.

Known copper-chelating agents (such as D-PA) have been investigated for their capacity to control angiogenesis and thus by inference, to impair cancer growth and metastasis. However, methanobactins and pharmaceutical compositions described herein have not been elucidated for cancer treatment before. Further provided herein is therefore the use of methanobactins and pharmaceutical compositions described herein for treatment of various cancers, including without limitation, reticulum cell sarcoma, bronchogenic and laryngeal squamous cell carcinomas, cervical, breast, colorectal, stomach, lung cancers, liver cancer, prostate cancer, brain cancer, chronic lymphoid leukemia, non-Hodgkin's lymphoma, multiple myeloma and Hodgkin's lymphoma.

### Neurodegenerative Disorders

Protein aggregation is a notable feature of various neurodegenerative disorders, including Parkinson disease, Alzheimer disease, Prion Disease including Creutzfeldt-Jakob disease (CJD), fatal familial insomnia (FFI), and Gerstmann-Straussler-Scheinker syndrome (GSS), familial amyotrophic lateral sclerosis (fALS) and many others. An increasing number of studies suggest that transition metals are able to accelerate the aggregation process of several proteins found in pathological deposits, and that in particular copper produces a most remarkable acceleration of aggregation. Hence, copper depletion by methanobactin treatment is therefore contemplated to reduce protein aggregation, thereby alleviating or even revert signs and symptoms of the disease.

It is therefore further envisaged to use methanobactins and pharmaceutical compositions described herein for treatment of neurodegenerative diseases including Parkinson Disease, Alzheimer Disease, Prion Disease, Huntington Disease and fALS.

### Diabetes

Moreover, defective copper regulation has been suggested as a causative mechanism of organ damage in diabetes which has been attributed to impaired anti-oxidant defence mechanisms and oxidative stress. Strikingly, TETA treatment was shown to act on mitochondrial proteins with roles in energy metabolism in diabetes patients, and resulted in restoration of cardiac structure and function (Jüllig et al., Proteomics Clin Appl. 2007 April; 1(4):387-99). As demonstrated in example 3 of the present application, methanobactins are surprisingly capable of efficiently removing accumulated mitochondrial copper—and are therefore, too, promising agents for a novel diabetes therapy based on the depletion of excess copper levels, particularly from the mitochondria, thereby reducing overall oxidative stress and tissue damage. In line with previous studies, methanobactins are particularly envisaged to improve diabetic cardiomyopathy and arterial and/or renal structure/function and to ameliorate left-ventricular (LV) hypertrophy in diabetic patients (see Zhang et al. Cardiovasc. Diabetol. 2014 Jun. 14; 13:100).

### Other Disorders

Further disease and disorders eligible for treatment with methanobactin and pharmaceutical compositions described herein comprise bacterial infections, inflammatory diseases, fibrosis, cirrhosis, lead and/or mercury poisoning.

In particular, during bacterial infections macrophages release copper in an attempt to kill invading microbes through copper toxicity. This leads to the induction of copper stress responses in invading microbes (Gleason et al., PNAS 2014 April; vol. 111, no.16:5866-5871). According to Gleason et al. (2014) this high level of host copper is favorable for SODS activation of C. albicans. C. albicans is the most prevalent human fungal pathogen—a yeast fungus—, which is able to combat the host immune response (e.g. macrophages) with its expressed superoxide dismutase 5 (SOD5), a monomeric copper-only SOD. Depletion of copper is therefore more importantly to reduce overall SODS activity, thus reducing human fungal pathogens during bacterial infections. In accordance, methanobactins and pharmaceutical compositions described herein are also envisaged for treatment of human fungal pathogens during bacterial infections such as C. albicans, which overexpresses SODS.

Therefore, the present invention encompasses a pharmaceutical composition, wherein bacterial infections are favorable for human fungal pathogens, preferably said human fungal pathogen is Candida albicans.

### Treatment

The term “treatment” in all its grammatical forms includes therapeutic or prophylactic treatment of the diseases described herein, in particular WD. A “therapeutic or prophylactic treatment” comprises prophylactic treatments aimed at the complete prevention of clinical and/or pathological manifestations or therapeutic treatment aimed at amelioration or remission of clinical and/or pathological manifestations. The term “treatment” thus also includes the amelioration or prevention of the diseases described herein, specifically WD.

The terms “subject” or “individual” or “animal” or “patient” are used interchangeably herein to refer to any subject, particularly a mammalian subject, for whom therapy is desired. Mammalian subjects include humans, non-human primates, dogs, cats, guinea pigs, rabbits, rats, mice, horses, cattle and the like, with human subjects being particularly envisaged for treatment according to the invention.

### Dosage

The exact dose of methanobactin may depend on the purpose of the treatment (e.g. prophylactic or maintenance therapy vs. treatment of acute WD), and will be ascertainable by one skilled in the art using known techniques. Adjustments for route of administration, age, body weight, general health, sex, diet, time of administration, drug interaction and the severity of the condition may be necessary, and will be ascertainable with routine experimentation by those skilled in the art. In general, dosages of 1 mg/kg body weight (bw) may be capable of eliciting the desired therapeutic effect as described elsewhere herein. Exemplary dosages applicable in the uses and methods of the invention include doses between 1 mg/kg bw and 1000 mg/kg bw, such as between 1 mg/kg bw and 100 mg/kg bw, and particularly between 1 mg/kg bw and 50 mg/kg bw, such as 1, 2, 5, 10, 15, 20, 25, 30, 35, 40, 45 or 50 mg/kg bw.

### Kit

It is also envisaged that methanobactins, in particular in stabilized form, and pharmaceutical compositions described herein can be provided as part of a kit. Accordingly, in a further aspect, the present invention also relates to a kit comprising methanobactins, specifically such in stabilized form, or pharmaceutical compositions comprising the same for the use of treatment of Wilson Disease, wherein the treatment reduces (i) whole liver copper levels, (ii) overall hepatocyte copper levels and/or (iii) hepatocyte mitochondrial copper levels.

The kit may be a kit of two or more parts, and comprises the methanobactins described previously, or a pharmaceutical composition comprising the same, and further active agents and/or pharmaceutical excipients. For instance, the kit may comprise one or more active agents or pharmaceutical compositions comprising the same useful for treating WD, such as d-penicillamine (D-PA), trientine (TETA) and tetrathiomolybdate (TTM), and/or zinc salts. The kit components may be contained in a container or vials. It is envisaged that the kit components are administered simultaneously, or sequentially, or separately with respect to the administration of the methanobactins or pharmaceutical compositions comprising the same. The present invention further encompasses the application of the kit components via different administration routes. E.g., conventional copper chelators may be administered orally, whereas the parenteral route of administration can be used for methanobactins.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

The term “less than” or “greater than” includes the concrete number. For example, less than 20 means less than or equal to. Similarly, more than or greater than means more than or equal to, or greater than or equal to, respectively.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### EXAMPLES

### Materials and Methods

### Animals

The Atp7b−/− rat strain (LPP rats) was originally provided by Jimo Borjigin (University of Michigan, Ann Arbor, USA) and further bred at the animal facility of Helmholtz Munich (Germany) or at the Nonclinical Research Center, Qu-BEST BIO Co., Ltd. (South Korea). Animals were on a 12 h light/dark cycle. Temperature and relative humidity were 22±1° C. and 50±5%, respectively, according to the European Convention 2007/526 EC. Animals were maintained on Altromin 1314 diet (Altromin Spezialfutter GmbH, Germany) containing 13 mg/kg copper (ad libitum). All animals were treated under the guidelines for the care and use of laboratory animals of the Helmholtz Munich. Heterozygous Atp7b+/− rats served as controls in this study unless otherwise stated.

### Animal Treatments

Animal experiments were done in accordance with the EU Animal Welfare Act and approved by the government authorities on animal care (Germany: permit No: 55.2-1-54-2532-65-17 from the Government of Oberbayern; South Korea: permit No: QBIACUC-A21199 and QBIACUC-A21199-2).

### Metabolic Profiling of Rats

Rats were housed individually in metabolic cages (Techniplast, Germany) for 4 days. Body weight change and food consumption were followed during treatment in 24-hour periods. Urine and feces of each rat was collected in 24-hour periods at 24, 48, 72 and 96 hours after treatment start. Feces was separated from chow residues and stored at −20° C. Urine samples were cleared by centrifugation step stored at 4° C. for subsequent metal content determination.

### In Vivo Treatments

Experiments shown in FIGS. 4, 5A, B, D-F, 6A, 7 and FIGS. 9, 11-13: Atp7b−/− rats were intraperitoneally injected with either MB-OB3b (1154.26 g/mol, twice daily at 150 mg/kg bw, i.e. 130 μmol/kg), ARBM101 (851.2 g/mol twice daily at 110 mg/kg bw, i.e. 130 μmol/kg) or DPA (149.21 g/mol, once daily at 100 mg/kg bw, i.e. 670 μmol/kg). Treatments were typically started in still healthy (H) animals around 80 days of age. Animals were either treated at 4 consecutive days, or twice for 4 consecutive days with interjacent 3 days resting period. The latter dosing constituted one “standard” treatment cycle in the long-term studies, whereat animals were sacrificed upon 5 such cycles after an additional 2-3 weeks' drug-free observation period. Experiments shown in FIGS. 5C, 6B-D: Atp7b−/− rats (around 100 days of age) were intravenously injected into the tail vein once daily with ARBM101 at doses ranging from 25-200 mg/kg bw or per os with DPA (100 mg/kg bw) for 9 consecutive days.

### Histological Examination, Plasma/Serum AST and Bilirubin

Liver tissues were fixed overnight at 4° C. in 4% paraformaldehyde (PFA) and embedded in paraffin. Tissue samples were cut into 4 μm-thick sections and stained with hematoxylin and eosin for analyses. Blood samples were either taken sublingually or from the jugular vein. Therefrom Plasma AST, ALT and bilirubin was determined either with a Reflotron system (Roche) or an automated chemistry analyzer (AU480, Beckman Coulter, USA). Serum preparation was performed from blood samples collected through vena cava after final bleeding and directly subjected to metal analysis.

### Mitochondrial Analyses

Rat liver mitochondria were isolated from freshly prepared liver homogenates, and purified by differential and density gradient centrifugation using Nycodenz® (Axis-Shield). Freshly prepared liver mitochondria were used for analysis of ATP production using the ATP Bioluminescence Assay Kit (Roche Diagnostics, Mannheim, Germany) according to the manufacturer's guideline.

### Chelation Potency of Chelators on Isolated Mitochondria

Freshly isolated Atp7b−/− rat liver mitochondria were subjected to chelator treatments for 30 min with either 2 mM D-PA, MB-OB3b or ARBM101 and subsequently re-purified by a Nycodenz®-gradient. Mitochondrial copper levels were analyzed by ICP-OES (Ciros Vision; SPECTRO Analytical Instruments) after wet-ashing of samples with 65% nitric acid (Merck). Copper levels of buffer treated Atp7b−/− rat liver mitochondria were set to 100%.

### Cell Culture

HepG2 cells were kept in MEM with 2% FCS. We found that Zn-MB is time stable at 37° C. in contrast to metal-free methanobactin (FIG. 9C). Therefore, Zn-MB, generated by preparing a 20 mM MB solution and adding an equimolar concentration of Zn solution under pH control, was used in cell culture experiments. The rat hepatocellular carcinoma cell line HepG2 from ATCC was cultured at 37° C. in an atmosphere with 95% humidity and 5% CO2 in Dulbecco's Modified Eagle Medium (DMEM) with Glutamax and 1 g/L glucose, supplemented with 10% FCS Superior (Bio&SELL, Germany) and 1% Penicillin/Streptomycin (Gibco, United Kingdom). Cell number was determined either with a Neubauer counting chamber or with an automated cell counter (LUNA-II, Logos Biosystems). For treatments, 2×106 HepG2 cells were seeded into 6 cm plates and incubated for 24/48 h. Cells were treated with MB-OB3b, ARBM101 and DPA at doses from 0.2-1 mM. The copper histidine (Cu-His) stock solution was prepared. For copper uptake experiments, HepG2 cells were seeded into 6 cm plates and treated 24/48 h with increasing Cu-His doses.

2×10⁴ cells were seeded in 96-well plates and incubated for 24/48 h in the presence or absence of test substances. Cell toxicity was either measured by the CellTiter-Glo® Luminescent Cell Viability Assay (Promega, Germany), Hoechst or by the Neutral red cell toxicity assay. Carbonyl cyanide 3-chlorophenylhydrazone (CCCP, 0.25 mM) served as mitochondria toxic positive control.

HepG2 cells were pretreated with 0.5 mM Cu-His for 24 h and subsequently treated with 1 mM ARBM101 or DPA for 24 h. Cells were washed two times, counted and subjected to metal analysis.

### Metal Content Determination

Copper in liver homogenates, cell lysates and mitochondrial preparations were analyzed by ICP-OES (Ciros Vision, SPECTRO Analytical Instruments GmbH) after wet ashing of samples with 65% nitric acid (Zischka et al., log. cit.).

### Copper Chelators and Chemicals

Methanobactin OB3b (MB-OB3b) was isolated from Methylosinus trichosporium OB3b and MB-SB2 (recently termed ARBM101) from a proprietary strain. D-PA was a gift from Firma Heyl Pharma. CCCP was acquired from Sigma-Aldrich (Taufkirchen, Germany).

### Statistics

Data are represented as either individual (for LT animals) or mean values with standard deviation(SD). “N” represents number of animals/biological replicates and “n” the number of technical replicates. Statistical significance was analyzed using either 1-way ANOVA with or without multiple comparison test or by using unpaired two-tailed t-test. The data analyses were performed using GraphPad Prism 8 (GraphPad Software Inc., United States). Statistical differences are expressed as *p<0.05, **p<0.01, ***p<0.001, and ****p<0.0001.Example 1: MB-OB3b and ARBM101 efficiently deplete excess copper in vitro and in vivo.

### Example 1: MB-OB3b and ARBM101 Efficiently Deplete Excess Copper In Vitro and In Vivo.

When applied in vitro, both MB-OB3b and ARBM101 efficiently deplete copper from isolated copper burdened WD rat liver mitochondria and from copper loaded HepG2 hepatic cells (FIGS. 3A, 3B). They do so with no signs of overt cell toxicity up to millimolar concentrations (FIGS. 3C, 8A-8C). Importantly, in contrast to DPA, ARBM-101 restores the normal physiological pathway of copper excretion by binding profoundly to and rapidly mobilizing overloaded copper through the bile/fecal axis, as evidenced by an immediate and steady copper excretion into the feces in WD rat studies (FIGS. 4A, 9B).

### Example 2: MB-OB3b and ARBM101 Cause Successful Fecal Copper Excretion

Methanobactins cause significant and rapid faecal copper excretion in WD rats housed in metabolic cages (FIG. 4A). Untreated control and WD rats excrete around 3 μmol copper into faeces in 24 h (+/− control rats: 2.98±0.3 μmol/24 h; −/− WD rats: 3.44±0.5 μmol/24 h). This amount increases two to threefold upon either MB-OB3b or ARBM101, but not upon DPA treatment (FIG. 4A). In contrast, DPA routes significant but minor copper amounts into urine (˜0.5 μmol/24 h versus ˜0.1 μmol/24 h background excretion in untreated controls, FIG. 4B).

With respect to an animal copper balance, only MB-OB3b and ARBM101 cause negative daily copper balances of ˜120% (˜3.4 μmol/24 h) or ˜200% (˜6 μmol/24 h), respectively (FIG. 4C). This negative balance cannot be explained by a lower nutritional intake that was comparable in untreated vs. treated WD rats (FIG. 4D). Overt signs of toxicity were absent, as treated animals behaved normal and did not show weight loss (FIGS. 4E, 9A). Of note, while fecal copper excretion seemed to dampen somewhat within four consecutive MB-OB3b treatment days, this was less pronounced for ARBM101 (FIG. 4A). Thus, MBs cause a negative copper balance (FIGS. 4C, 9B) due to enormous copper (but not zinc FIG. 9C) mobilization via the physiological excretion route into feces, that was sustainable over days of treatment in the case of ARBM101.

### Example 3: ARBM101 Treatment Depletes Excessive Liver Copper

Four days of twice daily ARBM101 treatments significantly depleted liver copper (FIG. 5A), from both, the cytosolic and organelle compartments (FIG. 5A). Given the net fecal copper excretion of ˜380 μg/24 h (FIG. 4C), a WD rat liver of around 8 g and its copper content of ˜44 μmol copper (350 μg/g wet weight at 80 days of age) should be copper depleted by ARBM101 within 7-8 days of such twice daily treatment. In almost exact agreement with this rationale, liver (and liver subfractions) copper values dropped down to values typically observed in unaffected wild-type controls (FIG. 5B). Thus, a quantitative removal of copper from the liver is possible by intense ARBM101 treatments and does occur in a dose dependent fashion (FIG. 5C). This profound copper depletion alleviated cellular copper stress, as protective metallothionein levels returned to normal as well (FIG. 5D).

Importantly, all treated animals stayed healthy and no signs of a potential critical shortage of liver copper that would cause bioenergetic deficits were noticed. In fact, the contrary was observed, as the bioenergetic ATP production capacity of hepatocyte mitochondria from ARBM101-treated WD rats was fully restored to wild-type control levels (FIG. 5E) and mitochondria appeared with intact structures (FIG. 5F). Furthermore, while excess liver copper was most efficiently depleted into feces, no or only very minor changes in copper levels versus untreated healthy WD rats were observed in either serum, brain, kidney, heart (FIG. 6A), testis and spleen (data not shown). Thus, it is the excess liver copper that is depleted but not the essential copper in other organs.

### Example 4: Methanobactin Treatments Do Not Deplete Essential Systemic Copper but Ensure Prolonged Animal Survival With Normal Body Weights

In untreated WD rats, liver copper continuously accumulates leading to hepatitis onset around 90-100 days of age as diagnosed by weight gain stagnation, elevated serum liver enzymes (ALT>100 U/I, AST>200 U/I) Subsequently, animals fall clinically ill and show weight loss, strongly elevated serum liver enzymes and detectable serum bilirubin levels (>0.5 mg/dl, FIG. 10A). In this diseased stage, elevated (vs. levels in still healthy WD rats) serum copper (FIG. 6A), paradoxical serum ceruloplasmin activity (FIG. 10B) and elevated (vs. levels in still healthy WD rats) kidney copper (FIG. 6A) are found. Animals die typically around 20-30 days after hepatitis onset.

Based on these pathognomonic features, 9-days of daily intravenous ARBM101 injection delayed hepatitis onset/progression, as assessed by ALT reduction and weight gain, in a dose-dependent fashion and in contrast to untreated or DPA treated animals (FIG. 6B, C). Most importantly, ARBM101 prolonged survival of WD rats in a dose dependent fashion (FIG. 6D).

### Example 5: A New Treatment Regimen in WD Rats

Five intraperitoneally ARBM101 treated WD rats (16 single doses at 8 days in total) were closely monitored for animal weight, behaviour and serum liver transaminases. All animals demonstrated an intact health status for about 74 subsequent days, whereupon serum liver transaminases rose again (FIGS. 7A, 7B and 11). Thus, upon profound copper depletion by one ARBM101 treatment cycle of eight treatment days (FIG. 5B), the time to hepatitis onset almost exactly doubled, further validating the direct causal link of excess liver copper and hepatitis in WD rats.

To further investigate whether intense liver copper depletion by ARBM101, if repeatedly admitted, ensures long-term animal health, these animals were each subjected to a total of five such cycles of ARBM101 treatment and pause. Thereupon experiments were stopped after an additional three-week observation period (FIGS. 7A, 11). Treatment pause duration was determined by closely monitoring animal behaviour and weight and especially serum liver transaminases (FIGS. 7A, 7C and 11). Upon a re-rise of ALT/AST values to threshold values indicating re-occurring hepatitis onset, the next treatment cycle was initiated with the aim to keep the animals healthy throughout the whole experiment. As a result, upon experiment termination, healthy animal lifetime was between 400-500 days (FIGS. 7A and C), i.e. around four times the lifetime of untreated WD rats that die at around 120 days of age (FIG. 6D). Moreover, weight development in these long-term treated WD rats was highly comparable to wild type animals (FIG. 7C) and mating of a long-term treated WD male rat with a heterozygous female, gave rise to healthy offspring with normal litter size, indicating no decrease in reproduction capability. Liver and liver subfractional copper levels were still low in these long-term treated WD rats three weeks after the last treatment cycle, significantly lower as in diseased WD rats, in agreement with their healthy status (FIG. 7D). Liver sections of long-term ARBM101 treated WD rats exhibited typical features of still healthy WD rats (i.e., anisokaryosis, sporadic inflammatory infiltrates, ballooning, microvesicular vacuolation and irregularly stippled cytoplasm (FIGS. 7E, 12). In contrast to diseased WD rats, multifocal hepatocyte apoptosis as well as eosinophilic protein-insudations and hepatocytes with cleared cytoplasm were absent (FIGS. 7E and 12). In addition, mitochondrial structures, whether isolated or in situ, of long-term treated animals appeared normal, i.e. without characteristic copper related structural peculiarities (FIG. 13). Thus, none of the long-term treated WD rats presented signs of overt copper overload or hepatitis. Throughout their live, only one animal had a health issue causing weight loss at the very end of the experiment, plausible due to an ingested foreign particle, and neither related to WD pathology nor to treatments.

Importantly, none of these long-term treated animals experienced a day of clinical apparent WD liver disease (FIGS. 7 and 11-13).

In conclusion, the enormous liver copper depletion achieved by methanobactins brought liver copper very close to physiological levels and allowed for a new treatment regimen consisting of intense treatment cycles followed by prolonged drug holidays that ensured a healthy animal life. It is MBs unique combination of immense copper affinity, inertness of bound copper, and sustained mobilization that results in an unprecedented net copper excretion. Importantly, no compound related toxicities were observed, neither in vitro nor in vivo, and no signs of immunological complications were observed in the liver tissues from the long-term treated animals. Treatments caused fully restored mitochondrial power and constant animal weight gain.

1. A method of treating Wilson Disease in a subject, the treatment comprising at least one treatment cycle of (a) a first phase of a copper-binding methanobactin administration followed by (b) a second phase of non-treatment, wherein the second phase exceeds the first phase, wherein said methanobactin is a Methylosinus trichosporium OB3b methanobactin (mb-OB3b) or a Methylocystis sp. SB2 methanobactin (mb-SB2).

2. The method of claim 1, wherein the first phase lasts for a period of 1, 2, 3, 4, 5, 6, 7, 8, 9, 10 or more consecutive days.

3. The method of claim 1, wherein the methanobactin is administered in single doses once daily, twice daily, three times daily, four times daily, every other day or continuously.

4. The method of claim 1, wherein the second phase lasts for a period of at least about 1 week, 2 weeks, 3 weeks, 4 weeks, 5 weeks, 6 weeks, 7 weeks, 8 weeks, 9 weeks, 10 weeks or more.

5. The method of claim 1, wherein the second phase of said treatment cycle is followed by at least one further treatment cycle.

6. The method of claim 1, wherein the method of treatment comprises recurrent treatment cycles.

7. The method of claim 1, wherein Wilson Disease comprises acute phase Wilson Disease.

8. The method of claim 7, wherein acute phase Wilson Disease is characterized by acute liver failure.

9. The method of claim 1, wherein the methanobactin is administered in a dosage of at least 1 mg/kg body weight to the subject.

10. The method of claim 1, wherein said mb-OB3b is of the formula R¹GSCYR²SCM (II), wherein R¹ is selected from (N-2-isopropylester-(4-thionyl-5-hydroxy-imidazole) and N-2-isopropylester-(4-thiocarbonyl-5-hydroxy-imidazolate), and R² is selected from pyrrolidine-(4-hydroxy-5-thionyl-imidazole) and pyrrolidine-(4hydroxy-5-thiocarbonyl-imidazolate), and mb-SB2 is of the formula R¹ASR²AA (III) wherein R¹ is 4-guanidinobutanoyl-imidazole and R² is 1-amino-2-hydroxy-oxazolone.

11. The method of claim 1, wherein said mb-OB3b has the formula (IV) 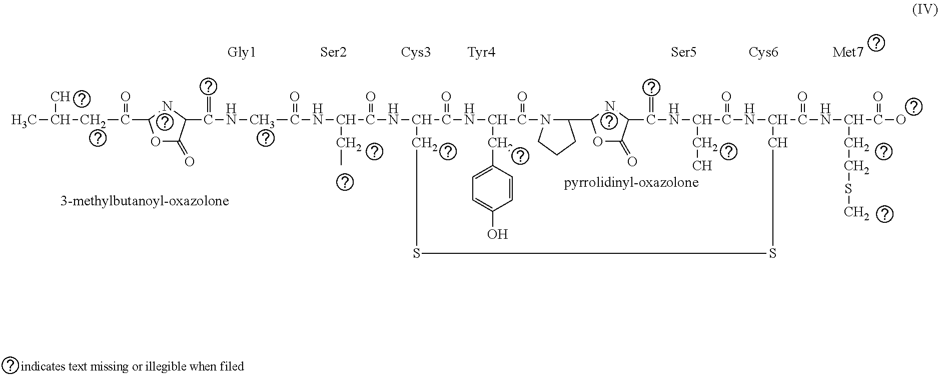 [SMILES: C=C(NCC(=O)NC(CC)C(=O)NC1CSSCC(C(=O)NC(CCSC)C(=O)O)NC(=O)C(CC)NC(=C)C2NC(OC2=O)C2CCCN2C(=O)C(Cc2ccc(O)cc2)NC1=O)C1NC(C(=O)CC(C)C)OC1=O]
or said mb-SB2 has the formula (V)

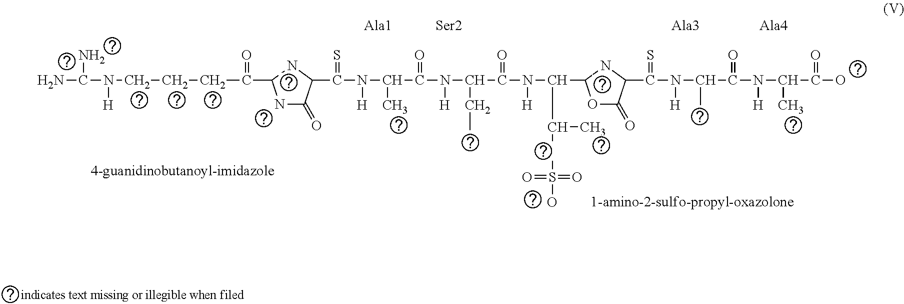 [SMILES: CCC(NC(=O)C(C)NC(=S)C1NC(C(=O)CCCNC(N)N)NC1=O)C(=O)NC(C(C)CS(=O)(=O)O)C1NC(C(=S)NC(C)C(=O)NC(C)C(=O)O)C(=O)O1]

12. The method of claim 1, wherein said mb-OB3b comprises or consists of the following structure (VI) 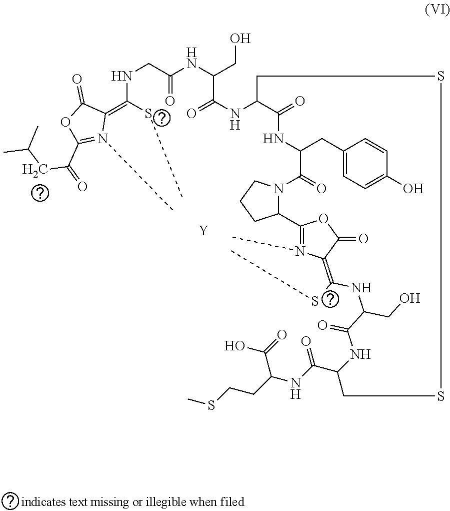
wherein Y is selected from Zn(I), Zn(II), Cu(I) and Cu(II);
or said mb-SB2 comprises or consists of the following structure (VII) 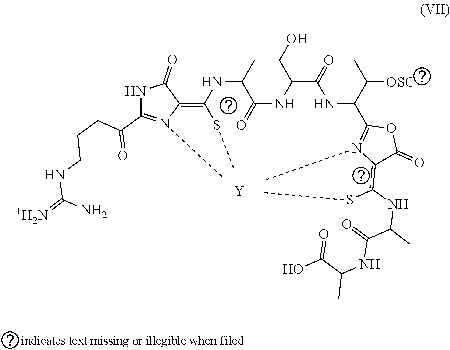
wherein Y is selected from Zn(I), Zn(II), Cu(I) and Cu(II).

13. The method of claim 1, wherein said methanobactin is provided in stabilized form.

14. The method of claim 1, wherein said methanobactin complexes Zn(I) and/or Zn(II) and/or is provided at a pH≥9.

15. The method of claim 1, wherein said methanobactin binds Cu(I) with a K_{d} of 10⁻¹⁵ or less and/or wherein treatment comprises at least one treatment cycle comprising administration of a methanobactin binding Cu(I) with a K_{d} of 10⁻¹⁵ or less, and at least one treatment cycle comprising administration of a methanobactin binding Cu(I) with a K_{d} of 10⁻¹⁵ or more.
